(19) United States
(12) Patent Application Publication
(10) Pub. No.: US 2024/0424467 A1
(43) Pub. Date: Dec. 26, 2024

(54) COOLING EFFLUENT OF OXIDATIVE DEHYDROGENATION (ODH) REACTOR WITH QUENCH HEAT EXCHANGER

(71) Applicant: NOVA CHEMICALS (INTERNATIONAL) S.A., Fribourg (CH)

(72) Inventors: Bolaji OLAYIWOLA, Calgary (CA); Vasily SIMANZHENKOV, Calgary (CA); Shahin GOODARZNIA, Calgary (CA)

(73) Assignee: NOVA CHEMICALS (INTERNATIONAL) S.A., Fribourg (CH)

(21) Appl. No.: 18/684,733

(22) PCT Filed: Aug. 16, 2022
(86) PCT No.: PCT/IB2022/057659

§ 371 Date: Feb. 19, 2024

Related U.S. Application Data
(60) Provisional application No. 63/237,000, filed on Aug. 25, 2021.

(51) Int. Cl. B01J 19/00; B01J 4/00; B01J 8/06; C07C 11/04

(52) U.S. Cl. CPC B01J 19/0013; B01J 4/002; B01J 8/067; C07C 11/04; B01J 2219/00054; B01J 2219/00076; B01J 2219/00087; B01J 2219/2409

(57) ABSTRACT

A system and method including an oxidative dehydrogenation (ODH) reactor system, feeding ethane and oxygen to an ODH reactor having ODH catalyst, and dehydrogenating ethane to ethylene via the ODH catalyst in presence of the oxygen in the ODH reactor, thereby forming acetic acid in the ODH reactor. The ODH reactor effluent is discharged through a quench heat exchanger, thereby cooling the effluent via the quench heat exchanger to below a temperature threshold, the effluent including ethylene, acetic acid, water, carbon dioxide, carbon monoxide, and unreacted ethane, wherein residence time of the effluent from the ODH reactor to effluent discharge outlet of the quench heat exchanger is less than a specified upper limit.

### CLAIM OF PRIORITY

This application claims priority to U.S. Provisional Application No. 63/237,000 filed Aug. 25, 2021, the entire contents of which are hereby incorporated by reference.

### TECHNICAL FIELD

This disclosure relates to oxidative dehydrogenation (ODH) to produce ethylene. More specifically, the disclosure relates to cooling effluent from an ODH process using a quench heat exchanger and with short residence times to limit formation of unwanted products prior to downstream processing.

### BACKGROUND ART

Catalytic oxidative dehydrogenation of alkanes into corresponding alkenes is an alternative to steam cracking. In contrast to steam cracking, oxidative dehydrogenation (ODH) may operate at lower temperature and generally does not produce coke. For ethylene production, ODH can provide a greater yield for ethylene than does steam cracking. The ODH may be performed in a reactor vessel having catalyst for the conversion of an alkane to a corresponding alkene. Acetic acid as a byproduct may be generated in the conversion of the lower alkanes (e.g., ethane) into the corresponding alkenes (e.g., ethylene).

The product alkene and byproduct acetic acid may each be recovered from the ODH reactor effluent. A premise has been that the ODH reactor effluent does not experience significant additional reaction while flowing through effluent discharge piping at the ODH reactor prior to being cooled.

### SUMMARY OF INVENTION

An aspect relates to a method of operating an oxidative dehydrogenation (ODH) reactor system, the method including feeding ethane, oxygen, and diluent to an ODH reactor having ODH catalyst. The method includes dehydrogenating ethane to ethylene via the ODH catalyst in presence of the oxygen in the ODH reactor, thereby forming acetic acid in the ODH reactor. The method includes discharging effluent from the ODH reactor through a quench heat exchanger, thereby cooling the effluent via the quench heat exchanger to below a temperature threshold. The effluent includes ethylene, acetic acid, water, carbon dioxide, carbon monoxide, and unreacted ethane. The residence time of the effluent from the ODH reactor to an outlet of the quench heat exchanger that discharges the effluent is less than a specified upper limit.

Another aspect relates to a method of an ODH reactor system, the method including providing feed including ethane and oxygen to an ODH reactor, and dehydrogenating ethane to ethylene via ODH catalyst in the ODH reactor. The method includes discharging effluent from the ODH reactor through a quench heat exchanger, thereby cooling the effluent via the quench heat exchanger to below a specified temperature threshold. The effluent comprises ethylene, acetic acid, water, carbon dioxide, carbon monoxide, and unreacted ethane. Residence time of the effluent from an outlet of the ODH reactor that discharges the effluent to an outlet of the quench heat exchanger that discharges the effluent as cooled is less than an upper limit as specified to decrease occurrence of an unwanted reaction in the effluent.

Yet another aspect relates to an ODH reactor system including an ODH reactor having ODH catalyst to dehydrogenate ethane in presence of oxygen to ethylene and generate acetic acid. The ODH reactor system includes a quench heat exchanger to cool effluent of the ODH reactor to below a threshold temperature. The effluent includes ethylene, acetic acid, water, carbon dioxide, carbon monoxide, and unreacted ethane. The ODH reactor system is configured to provide residence time of the effluent from an effluent outlet of the ODH reactor to an effluent outlet of the quench heat exchanger less than an upper limit as specified to decrease occurrence of an unwanted reaction in the effluent.

The details of one or more implementations are set forth in the accompanying drawings and the description below. Other features and advantages will be apparent from the description and drawings, and from the claims.

### BRIEF DESCRIPTION OF THE DRAWINGS

FIGS. 1-3 are block diagrams of ethylene production systems.

FIGS. 4-6 are diagrams of a direct attachment of the quench heat exchanger to the ODH reactor.

FIG. 7 is a diagram of a heat-pipe heat exchanger.

FIG. 8 is a block flow diagram of a method of operating an ODH reactor system.

FIG. 9 is a block diagram of the laboratory reactor system utilized to perform the Examples 1-5.

FIG. 10 is an image of a tube having fouling material in the tube.

Like reference numbers and designations in the various drawings indicate like elements.

### DESCRIPTION OF EMBODIMENTS

Aspects of the present disclosure are directed to dehydrogenating ethane to ethylene via an oxidative dehydrogenation (ODH) catalyst in presence of oxygen in an ODH reactor. Thus, aspects relate to an ODH reactor system in ethylene production. The ODH reactor system includes the ODH reactor that converts ethane to ethylene. Acetic acid may also be formed in the ODH reactor. The technique may include discharging a product effluent including at least ethylene and acetic acid from the ODH reactor.

A problem may be that unwanted reactions occur in the effluent while the effluent is at elevated temperature. In particular, the unwanted reactions may occur in the effluent flowing through effluent piping as discharged from the ODH reactor at or near the operating temperature of the ODH reactor. Therefore, to reduce presence of these unwanted reactions, the present techniques provide a solution of discharging the effluent from the ODH reactor through a quench heat exchanger that cools the effluent. In particular, the discharging of the effluent through the quench heat exchanger may involve discharging the effluent through an outlet nozzle of the ODH reactor vessel and passing the effluent from the ODH reactor nozzle through the quench heat exchanger.

The ODH reactor system includes the quench heat exchanger. The quench heat exchanger may cool the ODH reactor effluent without condensing components in the effluent. Advantageously, the quench heat exchanger may cool the effluent to below a temperature so that the undesired reactions in the effluent do not significantly occur.

In implementations, the ODH reactor system is configured to limit the residence time of the effluent at elevated temperature. In particular, the residence time of the effluent as discharged at reactor temperature from the outlet of the ODH reactor to the outlet of the quench heat exchanger (that discharges cooled effluent) may be limited. The ODH reactor system may be configured to give this residence time as less than a specified upper limit (threshold) in seconds (e.g., less than one minute). This upper limit of the residence time may be specified to decrease time of the effluent at elevated temperature and thus decrease occurrence of unwanted reactions in the effluent. These unwanted reactions may more readily occur at higher temperature, such as with the effluent at (or near) typical operating temperatures of the ODH reactor. Therefore, the shorter the time (the lower the residence time) of the effluent at these greater temperatures may reduce manifestation of the unwanted reactions.

Experimental work presented in the Examples below focused on thermal reactivity of ODH product gas (resembling ODH reactor effluent) in absence of ODH catalyst. This work identified the problem of considerable reactions within mixtures resembling ODH reactor effluent. Depending on the particular example, the mixtures mimicking (approximating) ODH reactor effluent included various respective combinations of components selected from ethylene, acetic acid, water, oxygen, carbon dioxide, and ethanol. The reactions in the mixtures included gas phase reactions and reactions as catalyzed by the metal inner-surface of the conduit (tube) through which the mixtures flowed. These reactions gave undesirable gas byproducts (primarily carbon monoxide and carbon dioxide) and solid fouling material rich in carbon and elemental oxygen.

In commercial implementations, the presence of these unwanted thermal reactions downstream of the ODH reactor may negatively affect ODH plant economics, for example, due to: (1) possible decrease in selectivity or yield of ethylene and acetic acid; and (2) possible increase in selectivity or yield of carbon monoxide (CO) and carbon dioxide (CO₂). Furthermore, the presence of oxygen/carbon rich-solid fouling may negatively impact operation of ODH plant due to potential for plugging piping downstream of the ODH reactor. Such fouling (plugging) could lead to an unwanted and costly shutdown of the ODH plant.

However, experimental work (such as in the Examples below) identified that these undesired reactions diminish as temperature of the product stream (effluent) is reduced to below about 275° C. or about 250° C. (but kept above the hydrocarbon and water dew point for operating reasons) and with relatively short time of the product stream (effluent) at higher temperature prior to being cooled. The Examples indicated that limiting the time to seconds (e.g., less than one minute) of the effluent at or near the typical temperature of the effluent as discharged from the ODH reactor may be beneficial.

With this in mind, embodiments of the present techniques place a quench heat exchanger downstream of the ODH reactor to cool the reactor effluent to below an upper temperature threshold, such as 200° C., 225° C., 250° C., 275° C., or 300° C. A lower temperature threshold to avoid is the dew point (e.g., 150° C.) of the mixture. The specified upper limit for residence time for the effluent from the outlet of the ODH reactor to the outlet of the quench heat exchanger may be, for example, 60 seconds, 40 seconds, 20 seconds, 10 seconds, 9 seconds, or 8 seconds, and the like.

For embodiments that consider the residence time of the ODH reactor effluent from the outlet of the ODH reactor to the outlet of the quench heat exchanger, the residence time may be the combination of the residence time of the effluent through the discharge piping from the ODH reactor plus the residence time of the effluent through the quench heat exchanger that receives the effluent from the discharge piping. The residence time of the effluent through the discharge piping may be the ratio of the inside volume of the discharge piping to the volumetric flow rate of the ODH reactor effluent through the discharge piping. The residence time of the effluent through the quench heat exchanger may be the ratio of an inside volume of the quench heat exchanger to the volumetric flow rate of the ODH reactor effluent through the quench heat exchanger. The quench heat exchanger in these embodiments may typically have a process side for the effluent and a utility side for the cooling medium. Thus, the residence time of the effluent through the quench heat exchanger may be the ratio of the inside volume of the process side of the quench heat exchanger to the volumetric flow rate of the effluent through the process side of the quench heat exchanger. In some implementations, the process side may be tubes (tube side) of the quench heat exchanger that receives the ODH reactor effluent from the discharge piping. The quench heat exchanger may have tubes (e.g., with the quench heat exchanger as a shell-and-tube heat exchanger) and in which the effluent flows through the tubes. Therefore, the residence time of the effluent through the quench heat exchanger may be the ratio of the inside volume of the tubes collectively (tube side) to the volumetric flow rate of the ODH reactor effluent through the tube side of the quench heat exchanger. In instances in which the ODH reactor effluent does not flow through the tubes but instead flows through the shell side (exterior of the tubes) of the quench heat exchanger as a shell-and-tube heat exchanger, the residence time of the effluent through the quench heat exchanger may be the ratio of the inside volume of the shell or shell side of the quench heat exchanger to the volumetric flow rate of the ODH reactor effluent through the shell side of the quench heat exchanger.

Residence time of a fluid through a vessel or conduit may be defined as the ratio of inside volume of the vessel or conduit to the volumetric flowrate (volume per time) of the fluid passing through the vessel or conduit. The volumetric flow rate (and thus the residence time) may vary as a function of operating pressure and operating temperature, including at a constant mass flow rate and at constant composition. The residence time is based on actual conditions of pressure and temperature. In contrast, the residence time in the Examples is not calculated based on actual conditions of pressure and temperature. Therefore, the residence time in the Examples is an approximate residence time. This approximate residence time at higher temperatures in the Examples was about 9 seconds, indicating an order of magnitude (e.g., less than one minute) for a desirable specified upper limit for actual residence time of the effluent at reactor discharge temperatures in commercial configurations.

For an ODH reactor platform, such as fixed bed, fluidized bed, moving bed, or swing bed, the quench heat exchanger can be disposed a short distance (e.g., less than 20 feet) downstream of the outlet of ODH reactor. The conduit (piping) conveying the reactor effluent to the quench heat exchanger can have a static internal (e.g., packing) to reduce flow volume and/or cross-sectional flow area of the conduit to decrease residence time through the conduit. In implementations, the static internal may be akin to a static mixer. Moreover, in some implementations, the inlet of the quench heat exchanger is attached directly (e.g., a flange-to-flange connection) to the outlet of the ODH reactor so to reduce residence time of the effluent at elevated temperature. The quench heat exchanger may be a shell-and-tube heat exchanger. In other implementations, the quench heat exchanger may have a heat pipe design, as discussed below.

In yet other implementations, the quench heat exchanger may be a quench vessel having internal nozzles to spray a cooling fluid (e.g., cooling liquid such as liquid water) in direct cooling of the effluent. This quench vessel may be labeled as a quench heat-exchanger vessel. The spray of the cooling liquid may be carried out such that the cooling liquid enters the gas phase (in the quench vessel) in atomized form (small liquid particles) to facilitate that the cooling liquid is vaporized so to avoid retention of cooling liquid in liquid form and avoid condensation of effluent components. For such a quench vessel with spray nozzles, heat is exchanged between the effluent and the cooling fluid in the direct cooling. For the cooling fluid as liquid, the heat of vaporization of the cooling liquid may contribute to the heat transfer in addition to cooling via latent heat. The quench vessel as a quench heat-exchanger vessel may be a vessel downstream of the ODH reactor vessel. In other implementations, the quench heat exchanger is spray nozzles disposed in an upper portion of the ODH reactor with the ODH reactor as a fluidized-bed reactor. Thus, in those implementations, the ODH reactor vessel may also be a quench vessel in having a non-condensing quench section in an upper (top) portion of the ODH reactor vessel.

For implementations with the ODH reactor as a fluidized-bed reactor, an option is to locate the quench heat exchanger (e.g., heat pipe design or spray nozzles) within a top portion (catalyst disengagement section) of the ODH reactor.

As discussed, advantages of employing the quench heat exchanger and with short residence time may include improvement in ODH plant economics by: (a) reducing or eliminating unwanted gas phase reactions (post-ODH reactor) that negatively affect ethylene selectivity/yield and acetic acid selectivity/yield; and (b) improve ODH plant operational reliability by reducing or eliminating unwanted oxygen/carbon rich-solid fouling and pluggage post-ODH reactor. The residence time of the product mixture gas from the ODH reactor through the quench heat exchanger may be specified below a threshold value to avoid formation of undesirable gas phase reactions and/or solid-based fouling. This residence time may be controlled or altered via the installation of the quench heat exchanger, such as in placement of the quench heat exchanger and installing piping internals in conduits or nozzles. The technique may include maintaining operating temperature of the quench heat exchanger below a threshold value, such as 200° C., 250° C. or 275° C.

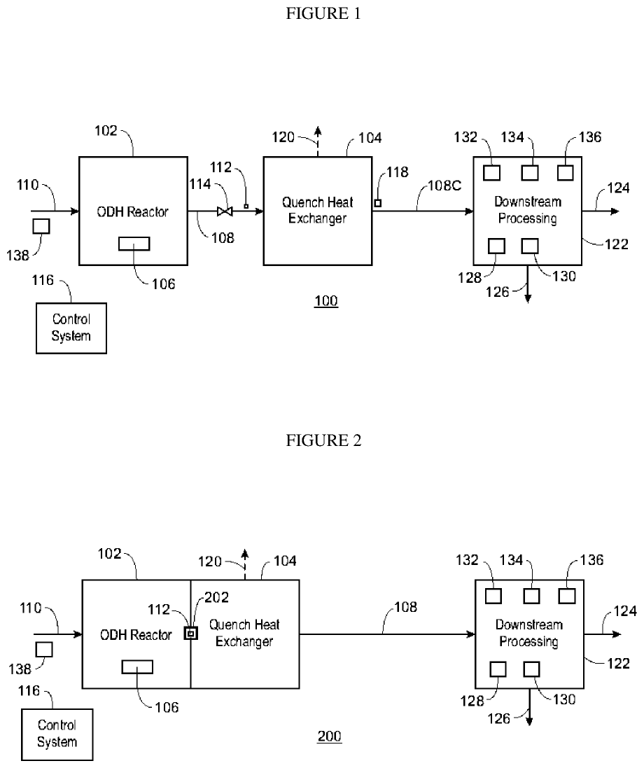

FIG. 1 is an ethylene production system 100 having an ODH reactor system. The ODH reactor system includes an ODH reactor 102 vessel and a quench heat exchanger 104. In operation, the ODH reactor 102 dehydrogenates ethane to ethylene (product) via an ODH catalyst 106 in presence of oxygen in the ODH reactor 102. Acetic acid (byproduct) may also be formed in the ODH reactor 102. The effluent 108 discharged from the ODH reactor 102 may include at least ethylene, acetic acid, water, carbon dioxide (CO₂), carbon monoxide (CO), and unreacted ethane. The effluent 108 may discharge from an outlet of the ODH reactor 102. The outlet may be labeled as an effluent outlet of the ODH reactor 102.

The feed 110 to the ODH reactor may typically include at least ethane and oxygen. To maintain the feed 110 mixture outside of flammability conditions (outside of the flammability envelope), the feed 110 mixture may be diluted. In other words, a diluent may be included in the feed 110. Examples of diluent that may be utilized include water (steam), nitrogen, CO₂, helium (He), argon (Ar), methane, etc., or mixtures thereof. In embodiments, water is the diluent. The water may be in the form of steam in the feed 110. Steam or vaporized water can be an attractive diluent, for example, due to the relative simplicity of the separation of the water from the ODH reactor product stream (effluent 108) in implementations. For embodiments with water employed as the diluent, the water in the effluent 108 may include both unreacted diluent water and water generated in the ODH reaction.

The ODH reactor 102 vessel has the ODH catalyst 106 to dehydrogenate ethane to ethylene. The operating temperature of the reactor 102 may be, for example, in the range of 300° C. to 450° C. The ODH reaction may typically be exothermic. The ODH reactor 102 system may utilize a heat-transfer fluid for controlling temperature of the reactor 102. In some embodiments, the heat-transfer fluid may flow through a heat-transfer jacket, such as a vessel jacket of the reactor 102 or a jacket internal in the reactor vessel 102. The heat-transfer fluid may be employed to remove heat from (or add heat to) the ODH reactor 102. The heat transfer fluid can be, for example, steam, water (including pressurized or supercritical water), oil, or molten salt, and so forth. The ODH reactor 102 may be, for example, a fixed-bed reactor (operating with a fixed bed of ODH catalyst) or a fluidized-bed reactor (operating with a fluidized bed of catalyst), or another reactor type. The ODH reaction of ethane (C₂H₆) to ethylene (C₂H₄) in the ODH reactor 102 via the ODH catalyst 106 may include or be C₂H₆+0.5 O₂→C₂H₄+H₂O. Additional reactions in the ODH reactor 102 may include:

C₂H₆+1.5O₂→CH₃COOH+H₂O

C₂H₆+2.5O₂→2CO+3H₂O

C₂H₆+3.5O₂→2CO₂+3H₂O

C₂H₄+O₂→CH₃COOH

C₂H₄+2O₂→2CO+2H₂O

C₂H₄+3O₂→2CO₂+2H₂O

CH₃COOH+O₂→2CO+2H₂O

CH₃COOH+2O₂→2CO₂+2H₂O

CO+0.5O₂→CO₂

Thus, in addition the ethylene formed, water (H₂O), acetic acid (CH₃COOH), carbon monoxide (CO), and carbon dioxide (CO₂) may also be formed in the ODH reactor 102. The effluent 110 can include unreacted diluent, which may be water in certain embodiments.

For the ODH reactor as a fixed-bed reactor, reactants (e.g., ethane and oxygen in the feed 110) may be introduced into the reactor at one end and flow past an immobilized catalyst (e.g., ODH catalyst 106). Products (e.g., ethylene, acetic acid, and other reaction products such as H₂O, CO, and CO₂) are formed and an effluent (e.g., effluent 110) having the products may discharge at the other end of the reactor. The fixed-bed reactor may have one or more tubes (e.g., metal tubes, ceramic tubes, etc.) each having a bed of the catalyst 106 and for flow of reactants. For the reactor 102, the flowing reactants may be at least ethane and oxygen. The tubes may include, for example, a steel mesh. Moreover, a heat-transfer jacket adjacent the tube(s) or an external heat exchanger (e.g., feed heat exchanger or recirculation heat exchanger) may provide for temperature control of the reactor 102. The aforementioned heat transfer fluid may flow through the jacket or external heat exchanger. Lastly, variations to the fixed-bed reactor, such as moving-bed reactor or swing-bed reactor (rotating-bed reactor), may be employed.

The ODH reactor as a fluidized bed reactor can be (1) a non-circulating fluidized bed, (2) a circulating fluidized bed with regenerator, or (3) a circulating fluidized bed without regenerator. In implementations, a fluidized bed reactor may have a support for the ODH catalyst. The support may be a porous structure or distributor plate and disposed in a bottom portion of the reactor. Reactants may flow upward through the support at a velocity to fluidize the bed of ODH catalyst. The reactants (e.g., ethane, oxygen, etc. for the reactor 102) are converted to products (e.g., ethylene and acetic acid in the reactor 102) upon contact with the fluidized catalyst. An effluent (e.g., effluent 110) having products may discharge from an upper portion of the reactor. A heat-transfer jacket (cooling jacket on the reactor vessel) may facilitate temperature control of the reactor. The fluidized bed reactor may have the jacket, heat-transfer tubes, or an external heat exchanger (e.g., feed heat exchanger or recirculation loop heat exchanger) to facilitate temperature control of the reactor. The aforementioned heat transfer fluid may flow through the reactor tubes, jacket, or external heat exchanger.

As indicated, the ODH catalyst 106 may be operated as a fixed bed or fluidized bed, and the like. Catalyst known for ODH of ethane may be employed as the ODH catalyst 106. In implementations, the ODH catalyst 106 composition may have little or no effect on the occurrence of the unwanted reactions in the ODH reactor effluent 108. An exception may be for an ODH catalyst 106 that produces a byproduct that increases the production of fouling or undesirable products while the effluent moves from the reactor 102 to and through the quench heat exchanger 104.

In certain embodiments, ODH catalyst 106 that can give an ODH reaction that dehydrogenates ethane to ethylene and forms acetic acid as a byproduct may be applicable to the present techniques. A low-temperature ODH catalyst may be beneficial. One non-limiting example of an ODH catalyst 106 that may be utilized in the ODH reactor 102 is a low-temperature ODH catalyst that includes molybdenum (Mo), vanadium (V), tellurium (Te), niobium (Nb), and oxygen (O), wherein the molar ratio of molybdenum to vanadium is from 1:0.12 to 1:0.49, the molar ratio of molybdenum to tellurium is from 1:0.01 to 1:0.30, the molar ratio of molybdenum to niobium is from 1:0.01 to 1:0.30, and oxygen is present at least in an amount to satisfy the valency of any present metal elements. The molar ratios of molybdenum, vanadium, tellurium, niobium can be determined by inductively coupled plasma mass spectrometry (ICP-MS). The catalyst may be low temperature in providing for the ODH reaction at less than 450° C., less than 425° C., or less than 400° C.

As discussed, associated with the ODH reaction that dehydrogenates the ethane, a byproduct formed may be acetic acid. As further mentioned, also formed byproducts associated with the ODH reaction may include water, CO₂, and CO. Thus, the effluent 108 discharged from the ODH reactor 102 vessel may include ethylene, acetic acid, water, CO₂. CO, unreacted ethane, and unreacted diluent (which may be water in embodiments). The temperature of the effluent 108 as discharged may be, for example, in the range of 300° C. to 450° C. commensurate with the operating temperature (e.g., 300° C. to 450° C.) of the reactor 102 vessel.

In the illustrated embodiment of FIG. 1, the effluent 108 may be routed through (transported by) a conduit from the ODH reactor 102 to the quench heat exchanger 104. The effluent 108 may enter the quench heat exchanger 104 through an inlet of the quench heat exchanger 104. The inlet may be labeled as an effluent inlet of the quench heat exchanger 104. In certain implementations, the size (e.g., nominal diameter or inside diameter) of the conduit conveying the effluent 108 from the ODH reactor 102 to the quench heat exchanger 104 may be specified smaller to reduce residence time of the effluent 108 through the conduit. However, a conduit having larger diameter may be desired for mechanical integrity or other reasons. In certain implementations, a static internal 112 (e.g., metal, ceramic, etc.) may be situated in the conduit (e.g., along the conduit) to reduce residence time of the effluent 108 in the conduit. The static internal 112 may be generally non-moving elements. The static internal 112 may be packing, such as generally spherical (e.g., metal or ceramic balls) or irregular shaped objects, fixed in the conduit. The static internal 112 may be packing fixed in the conduit, such as packing utilized in absorber, stripper, or distillation columns. The static internal 112 may be, for example, plates, baffles, or fixed helical-shaped objects. In a particular implementation, the static internal 112 is a static mixer (or multiple static mixers disposed linearly in series). The static internal 112 may reduce the volume available for flow in the conduit to decrease the residence time. The static internal 112 may reduce the cross-sectional area in the conduit available for flow to decrease the residence time in the conduit.

In some implementations, a valve 114 may be disposed along the conduit. In operation, the valve 114 may be normally open. The valve 114 can be, for example, a manual valve or an automated on/off valve. The valve 114 may be, for instance, an isolation valve to facilitate isolation of the quench heat exchanger 104 from the ODH reactor, such as for maintenance outside of normal operations, and the like.

The effluent 108 may be cooled in the quench heat exchanger 104 to below a specified temperature threshold that reduces unwanted reactions in the effluent 108. This temperature threshold may be, for example, 300° C., 275° C., 250° C., 225° C., or 200° C. The temperature may be maintained above the dew point of the effluent 108 mixture. The temperature value of the dew point may be entered into a control system 116 by a user (e.g., human operator). The control system 116 may direct operation of the quench heat exchanger 104. In implementations, the control system 116 may determine (e.g., calculate) the dew point correlative with (based on) the composition and pressure of the effluent 106 (e.g., at or near the inlet of the quench heat exchanger 104)

In some implementations, the quench heat exchanger 104 is operated to cool the effluent 108 to within a temperature range. The upper limit of the temperature range may be the aforementioned specified upper temperature threshold (e.g., 250° C.). The lower limit of the temperature range may be slightly above the dew point (e.g., 150° C.) of the effluent 108. A temperature of interest may be the temperature of the cooled effluent 108C as discharged from an outlet of the quench heat exchanger 104. The outlet may be labeled as an effluent outlet of the quench heat exchanger 104. The effluent downstream of the quench heat exchanger 104 is given the reference numeral 108C. In implementations, a temperature sensor 118 may be situated to measure the temperature of the effluent 108C as discharged from the quench heat exchanger 104. In particular, the temperature sensor 118 may be disposed on the quench heat exchanger 104 at or near the process (effluent) outlet of the quench heat exchanger 104, or disposed on a discharge conduit for the effluent 108C from an outlet of the quench heat exchanger 104. The temperature sensor 118 may be, for example, a thermocouple or a resistance temperature detector (RTD), such as a platinum RTD. If a thermocouple is employed, the thermocouple may be rest in a thermowell inserted into the conduit. A temperature transmitter (instrument transmitter operationally coupled to the temperature sensor 118) external to the quench heat exchanger 104 and the discharge conduit may send a signal indicative of the temperature as measured by the temperature sensor 118 to the control system 116. In implementations, the control system 116 may control (e.g., maintain, modulate, adjust, alter, etc.), for instance, the flow rate or temperature of the cooling medium or cooling fluid to the quench heat exchanger 104 to control the effluent 108C temperature, such as the temperature of the effluent 108C measured by the temperature sensor 118.

The control system 116 may facilitate or direct operation of the ODH reactor system (or the ethylene production system 100 more generally), such as operation of equipment, flow streams (including flow rate and pressure), and control valves. The control system 116 may receive data from sensors in the ODH reactor system. The control system 116, which may be or include multiple controllers, may perform calculations and receive or specify set points for control devices. The control system 116 may include a processor and memory storing code (e.g., logic, instructions, etc.) executed by the processor to perform calculations and direct operations of the system 100. The processor (hardware processor) may be more than one processor and may include a microprocessor, a central processing unit (CPU), a graphic processing unit (GPU), a controller card, circuit board, or other circuitry. The memory may include volatile memory (e.g., cache and random access memory), nonvolatile memory (e.g., hard drive, solid-state drive, and read-only memory), and firmware. The control system 116 may include a desktop computer, laptop computer, computer server, programmable logic controller (PLC), distributed computing system (DSC), controllers, actuators, or control cards. The control system 116 may receive user input that specifies the set points of control components in the ODH reactor 102 system. The control system 116 typically includes a user interface for a human to enter set points and other targets or constraints to the control system 116. In some implementations, the control system 116 may calculate or otherwise determine set points of control devices. The control determination by the control system 116 can be based at least in part on operating conditions of the system 100 including feedback information from sensors and transmitters, and the like.

In operation, the control system 116 may facilitate processes of the system 100 including to direct operation of the quench heat exchanger 104, as discussed herein. The control system 116 may facilitate maintaining the temperature (e.g., as measured by temperature sensor 118) of the cooled effluent 108C discharged from an outlet the quench heat exchanger 104 at a set point.

Furthermore, the control system 116 may implement the constraint that the effluent 108 is not cooled to below the dew point of the effluent 108. The dew point of the effluent 108 may be entered into the control system 116 by a user. Alternatively, the control system 116 may calculate the dew point of the effluent 108 or 108C correlative with the composition and pressure of the effluent 108 or 108C. The effluent 108 or 108C composition may be entered into the control system 116 by a user. Alternatively, the effluent 108 or 108C composition may be automatically indicated to the control system 116 from an online instrument analyzer (e.g., online gas chromatograph) that measures effluent 108 or 108C composition. The pressure of the effluent 108 in the quench heat exchanger 104 (or effluent 108C as discharged from the quench heat exchanger) may be indicated to the control system 116 from a pressure sensor. The pressure sensor may be disposed at or near the process (effluent) inlet of the quench heat exchanger 104, along the quench exchanger 104, or at or near the process outlet of the quench heat exchanger 104.

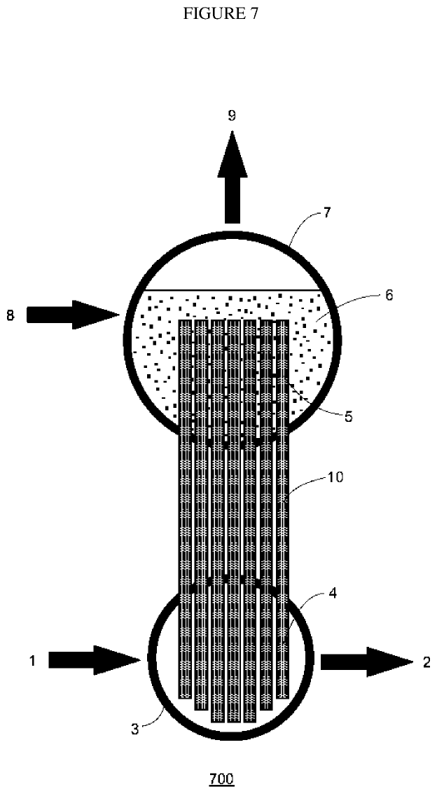

The quench heat exchanger 104 may be, for example, a shell-and-tube heat exchanger or a heat-pipe heat exchanger (see, e.g., FIG. 7), or a quench vessel having internal spray nozzles (e.g., atomizing spray nozzles) for spraying a cooling fluid (e.g., water) on the effluent 108, and so forth. For the quench heat exchanger 104 as a shell-and-tube heat exchanger, the cooling medium may flow through the tubes, or alternatively, through the shell. In implementations, the shell of the shell-and-tube heat exchanger may be characterized as a vessel. The cooling medium may be, for example, water, such as demineralized water, boiler feedwater, or steam condensate, and so on.

For the quench heat exchanger 104 as a heat-pipe heat exchanger (e.g., FIG. 7), the quench heat exchanger 104 may include two vessels. One vessel is a hot-section vessel in which the effluent 108 flows. The other vessel is a cold-section vessel having a liquid level of a cooling medium. Heat tubes or heat pipes (e.g., reference numeral 10 in FIG. 7) run from the interior of the hot-section vessel to the interior of the cold-section vessel. The cold end portion of the heat pipes in the cold-section vessel may be submerged in the liquid level of cooling medium (e.g., reference numeral 6 in FIG. 7). Heat transfer may occur from the effluent 108 along the heat pipes to the cooling medium. The temperature of the effluent 108 or 108C may be controlled, for example, by maintaining a specified temperature of the cooling medium and, therefore, maintaining a specified temperature differential between the cold-section vessel and the hot-section vessel. The temperature of the effluent 108 or 108C may be controlled in the heat exchanger having a heat pipe design, for instance, by controlling (adjusting) temperature of the cooling medium, as well as flow rate of the cooling medium and vaporization (if any) of the cooling medium in the cold-section vessel. The cooling medium in the cold-section vessel may be, for example, demineralized water, boiler feedwater, or steam condensate, and the like. The heat-pipe heat exchanger is known to one of ordinary skill in the art, as indicated, for example, in the international patent application having Publication Number WO 2017/141121 A1.

Typically, a heat pipe or tube includes a sealed tube (metallic) containing a working fluid and an internal capillary to transport the condensed working fluid from the end of the heat pipe in the cold section to the end of the heat pipe in the hot section. In operation, the working fluid in the heat tube is boiled or evaporated in the hot section taking heat from the hot fluid. The resulting vapor moves up the heat tube to the cold section. The vapor then condenses in the cold section giving up heat to the medium passing through the cold section. The resulting condensed liquid is transported through the capillary or wick in the tube to the hot section by gravity and capillary forces where it again evaporates.

In implementations, the quench heat exchanger 104 (e.g., as a shell-and-tube heat exchanger or heat-pipe design heat exchanger) may facilitate the generation of steam, as indicated by arrow 120. For the quench heat exchanger 104 as a shell-and-tube heat exchanger, the water as cooling medium is heated with heat from the effluent 108 to flash the water into steam. The steam generation system may include additional equipment, such as a vessel (e.g., flash vessel), a pump (e.g., boiler feedwater pump), etc. For the quench heat exchanger 104 as a heat-pipe heat exchanger, the water as cooling medium in the cold-section vessel may be vaporized generating steam, and the steam collected. As the cooling medium vaporizes, the cold-section vessel may be replenished with cooling medium to maintain the liquid level in the cold-section vessel.

The steam generated via the quench heat exchanger 104 may discharge into a steam header (or sub-header) conduit or through a conduit to a user, and so on. Higher-pressure steam may generally be more valuable than lower pressure steam. Higher-pressure steam, such as greater than 600 pounds per square inch gauge (psig) or greater than 1500 psig, may typically be more valuable than lower pressure steam, such as less than 600 psig or less than 150 psig. The pressure of the steam generated via the steam-generation heat exchanger 106 may be a function of the temperature of the effluent 108 driven by the operating temperature (ODH reaction temperature) of the ODH reactor 102.

The effluent 108C (as cooled by the quench heat exchanger 104) may discharge from the quench heat exchanger 104 to downstream processing 122. The aforementioned discharge conduit from the quench heat exchanger 104 may transport the cooled effluent 108C to the downstream processing 122. The downstream processing 122 may isolate product ethylene 124 and byproduct acetic acid 126.

The downstream processing 122 may include, for example, a separation system 128 to separate the majority of acetic acid and water as raw acetic acid from the effluent 108C. The separation system 128 may include, for instance, (1) a partial-condenser heat exchanger that cools the effluent 108C to condense acetic acid and water, and (2) a flash vessel that receives the effluent 108C from the partial-condenser. The flash vessel may recover the condensed acetic acid and condensed water in combination as raw acetic acid from the bottom portion of the flash vessel. The remaining portion of the effluent 108C may discharge overhead from the flash vessel. This remaining portion is generally gas (not liquid but can include vapor). In other implementations in lieu of a partial-condenser heat exchanger and flash vessel, the separation system 128 may instead be a quench tower that condenses the water and acetic acid in the effluent 108C, discharges the combination of the condensed water and condensed acetic acid as raw acetic acid in a bottoms stream, and discharges the remaining portion (not liquid) of the effluent 108C as an overhead stream.

The raw acetic acid may be provided to an acetic acid unit 130 to remove water from the raw acetic acid to recover product acetic acid 126 from the raw acetic acid. In implementations, the separation system 126 may discharge the raw acetic acid through a conduit to the acetic acid unit 130, e.g., such as to an extractor column in the acetic acid unit 130. Again, the raw acetic acid may be processed in the acetic acid unit 130 to remove water from the raw acetic acid to give acetic acid product 126 that is a coproduct of the ethylene production. The acetic acid product 126 may be, for example, at least 99 weight percent (wt. %) acetic acid. At least a portion of the water removed may be recovered as water product. In a particular implementation, the acetic acid unit 130 may include an extractor column (vessel) for injection of solvent to remove acetic acid, a water stripper tower (vessel) to process raffinate from the extractor column to recover water, and a solvent recovery column (vessel) to remove the solvent from the acetic acid discharged from the extractor column to give the acetic acid product 126.

The non-liquid portion of the effluent 108C discharged overhead from the separation system 128 may include water vapor, residual acetic-acid vapor, and gases such as ethylene, carbon dioxide, carbon monoxide, unreacted ethane, and other gases. In certain implementations, this non-liquid portion of the effluent 108C may flow to an acetic acid scrubber 132 (a vessel such as a column or tower) or similar vessel or system. The acetic acid scrubber 132 may scrub (remove) the acetic acid vapor and water vapor into a scrubbing liquid that discharges as a liquid bottoms stream. The acetic acid scrubber 132 may discharge overhead a process gas including the ethylene, carbon dioxide, carbon monoxide, unreacted ethane. In some instances, this process gas may be forwarded to a process gas compressor 134 (mechanical compressor) that increases the pressure of the process gas. The compressed process gas may be processed to remove light components, such as carbon monoxide and methane. The downstream processing may include a C2 splitter 136 that separates ethylene from ethane and discharges the product ethylene 124. The C2 splitter 136 may be a vessel that is a distillation column having distillation trays.

Lastly, the ethylene production system may include a feed heat exchanger 138 that heats the feed 110 to the ODH reactor 102. The feed heat exchanger 138 may be, for example a shell-and-tube heat exchanger or a plate-fin heat exchanger. In implementations, the feed heat exchanger 138 may be a cross exchanger with the effluent 108C to heat the feed 110 with the effluent 108C. For instance, the effluent 108C operationally between the quench heat exchanger 104 and the downstream processing 122 may be utilized to heat the feed 110 in the feed heat exchanger 138. In other implementations, the feed heat exchanger 138 may utilize steam instead of the effluent 108C as the heating medium.

FIG. 2 is an ethylene production system 200 that is similar to the ethylene production system 100 of FIG. 1, except that the system 200 has the quench heat exchanger 104 directly attached to the ODH reactor 102. Like reference numbers and designations in FIGS. 1 and 2 indicate like elements.

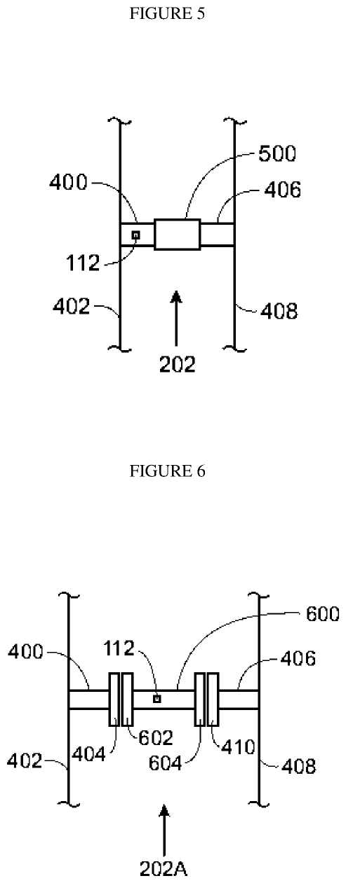

The direct attachment 202 (direct connection) of the quench heat exchanger 104 to the ODH reactor 102 may be implemented to give reduced residence time of the effluent 108 from the outlet (effluent discharge) of the ODH reactor 102 to the outlet (cooled effluent discharge) of the quench heat exchanger 104. The direct attachment 202 may be, for example, a flange-to-flange connection (e.g., FIG. 4). In other words, the flange of the outlet nozzle on the ODH reactor 102 may be bolted to the flange of the inlet nozzle on the quench heat exchanger 104. In other implementations, the direct attachment 202 may be a threaded connection (e.g., FIG. 5) (or welded connection) of the outlet nozzle on the ODH reactor 102 may to the inlet nozzle on the quench heat exchanger 104. The direct attachment 202 can include a static internal 112 (as discussed above), such as in the outlet nozzle of the ODH reactor 102 or in the inlet nozzle of the quench heat exchanger 104, or both. The presence of the internal 112 may reduce residence time of the effluent through the direct attachment 202. Lastly, a placement direct attachment can include a pipe fitting (e.g., pipe elbows), a short spool piece (pipe), valve, and the like, between the ODH reactor 102 outlet and the quench heat exchanger 104 inlet to account for disposition of the ODH reactor 102 vessel and the quench heat exchanger 104. Considerations with disposition may include, for example, the equipment footprint, different elevations of the respective nozzles, resolving physical interference between the ODH reactor 102 and the quench heat exchanger 102, and so on.

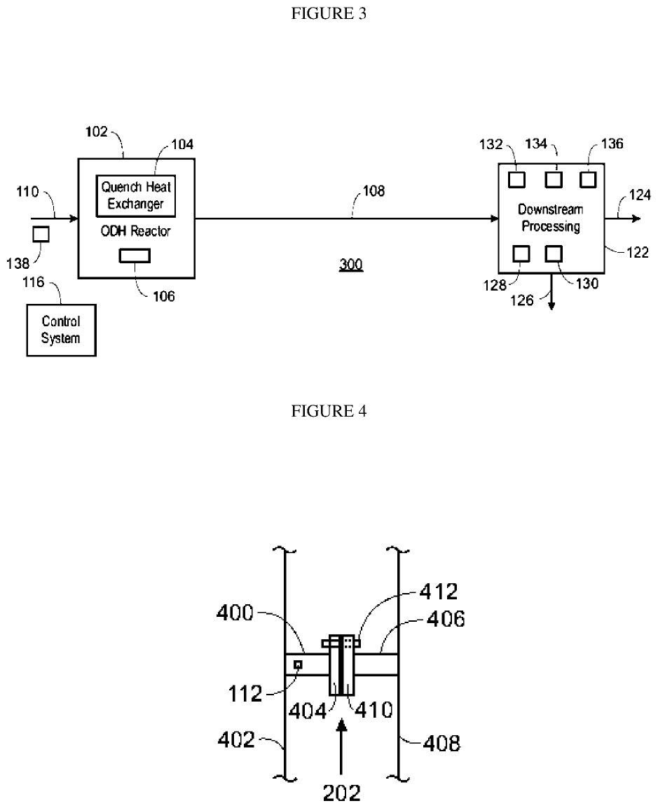

FIG. 3 is an ethylene production system 300 that is similar to the ethylene production system 100 of FIG. 1 and the ethylene production system 200 of FIG. 2, except that the system 300 has at least a portion of the quench heat exchanger 104 disposed in the ODH reactor 102. Like reference numbers and designations in FIGS. 1-3 indicate like or similar elements.

In the illustrated embodiment, the ODH reactor 102 may be, for example, a fluidized-bed reactor, and the quench heat exchanger 104 a heat-pipe heat exchanger or spray nozzles. The quench heat exchanger 104 is disposed in an upper portion of the ODH reactor 102, which in a fluidized bed reactor may be a disengagement section of the reactor 102. The disengagement section may be for disengagement of the fluidized catalyst from the product gas that discharges as effluent 108C from the upper portion of the ODH reactor 102 (fluidized-bed reactor in this embodiment).

For an implementation with the quench heat exchanger 104 as a heat-pipe heat exchanger, a hot portion of the heat-pipe heat exchanger is disposed in the ODH reactor 102 and a cold portion of the heat-pipe heat exchanger is disposed external to the ODH reactor 102. In other words, the hot-section vessel of the heat-pipe heat exchanger may be the ODH reactor 102 vessel (or vessel internal to the ODH reactor 102 vessel), and the cold-section vessel (having the cooling medium) of the heat-pipe heat exchanger may be a vessel external to the ODH reactor 102. In this embodiment, the hot end of the heat pipes of the heat-pipe heat exchanger are in the ODH reactor 102 and the cold end of the heat pipes are in the cold-section vessel external to the ODH reactor 102. Thus, the heat pipes may run from the interior of the ODH reactor 102 through a vessel wall of the ODH reactor 102, and then through the vessel wall of the cold-section vessel into the cooling medium in the cold-section vessel. In operation, heat transfer may occur from the reaction mixture in the ODH reactor 102 through the heat pipes to the cooling medium in the cold-section vessel of the heat-pipe heat exchanger.

In the illustrated embodiment of FIG. 3, for an implementation of the quench heat exchanger 104 as spray nozzles, the spray nozzles may be internal spray nozzles in the upper portion of the ODH reactor 102 (e.g., fluidized-bed reactor in this case). In operation, the spray nozzles as the quench heat exchanger 104 may spray cooling fluid (e.g., water such as demineralized water) onto the reaction mixture flowing upward as product gas in the disengagement section. Heat transfer may occur from the product gas or reaction mixture more generally to the sprayed stream or droplets of cooling medium. In implementations, the spray nozzles may be atomizing nozzles.

FIG. 4 depicts an implementation of the direct attachment 202 of FIG. 2 that is a flange-to-flange connection. In this depiction, an outlet nozzle 400 at the vessel wall 402 of the ODH reactor 102 (FIG. 2) has a flange 404. An inlet nozzle 406 at an exterior wall 408 of the quench heat exchanger 104 (FIG. 2) has a flange 410. If the quench heat exchanger 104 is a shell-and-tube heat exchanger, the inlet nozzle 406 may direct the effluent 108 (FIG. 2) into the shell side or, alternatively, into the tube side. The flanges 404, 410 are connected (bolted) together via multiple bolts 412. Only one bolt 412 is depicted for simplicity (clarity). A gasket may be installed between the bolted flanges 404, 410. Lastly, a static internal 112 (as described above) may be installed in one or both nozzles 400, 406 to shorten residence time of the effluent through the direct attachment 202. The static internal 112 may include more than one component or element. The direct attachment 202 may have more than one static internal 112.

FIG. 5 depicts an implementation of the direct attachment 202 of FIG. 2 that is a threaded connection. In this depiction, an outlet nozzle 400 at the vessel wall 402 of the ODH reactor 102 (FIG. 2) is not flanged. Similarly, an inlet nozzle 406 at the vessel or shell wall 408 of the quench heat exchanger 104 (FIG. 2) is not flanged. As in FIG. 4, if the quench heat exchanger 104 is a shell-and-tube heat exchanger, the inlet nozzle 406 may direct the effluent 108 (FIG. 2) into the shell side or, alternatively, into the tube side. In this example of a threaded connection, the respective mating ends of each nozzle 400, 406 have threads, and the threaded connection is via a pipe fitting 500, such as a threaded coupling. As in FIG. 4, a static internal 112 (or multiple static internals 112) may be installed in at least one of nozzles 400, 406 to shorten residence time of the effluent 108 through the direct attachment 202.

FIG. 6 depicts an example of a placement direct attachment 202A for the ODH reactor 102 to the quench heat exchanger 104. The placement direct attachment 202A includes placement piping 600 for the direct attachment. In implementations, the placement piping 600 may make feasible a direct attachment (e.g., as a placement direct attachment 202A). The placement piping 600 may facilitate avoiding physical interference between the ODH reactor 102 and the quench heat exchanger 104, and account for the elevation of the nozzle 400 being different than the elevation of nozzle 406, and so forth. The placement piping 600 may include a pipe spool piece (e.g., less than 3 feet in length). The placement piping 600 may include a pipe elbow(s) (e.g., 90° elbow) and other pipe fittings.

FIG. 7 is an example of a heat-pipe heat exchanger 700. A heat-pipe exchanger may have a hot section (routing the hot fluid stream to be cooled) and a cold section. The hot section and the cold section may have no common or adjoining external surfaces, wherein heat pipes extend from the interior of the hot section traverse the open space between the hot section and cold section and extend into the cold section. The heat pipes may include a wick and a capillary channel. The wick may be a porous metal substrate foam, felt, mesh, or screen.

The hot fluid 1 to be cooled may be representative of the effluent 108 as discharged from the ODH reactor (FIG. 1-2) or as the product gas in the disengagement section (FIG. 3) discharging as the effluent 108. The fluid 1 as cooled discharges from the heat-pipe heat exchanger as cooled fluid 2. The cooled fluid 2 may be representative of the effluent 108° C. as cooled by the quench heat exchanger 104 that is a heat-pipe heat exchanger in these embodiments.

The hot-section vessel 3 of the heat exchanger 700 may be representative of the hot-section vessel of the quench heat exchanger 104 downstream (FIGS. 1-2) of the ODH reactor 102. The depicted hot-section vessel 3 may be representative of the ODH reactor 102 vessel (or vessel internal to the ODH reactor 102 vessel) for the implementation (FIG. 3) of at least a portion of the quench heat exchanger 104 disposed in the ODH reactor 102.

The heat pipes 10 may each have internal capillaries and internal wicking. The working fluid in the heat pipe may include, for example, sodium, potassium, or cesium, or any combination thereof. The heat pipes 10 (heat tubes) may be, for example, stainless steel or other metal alloy including nickel and/or chromium.

The hot end 4 of the heat pipes 10 is in the hot-section vessel 3. The cold end 5 of the heat pipes is in the cold-section vessel 7. The hot-section vessel 3 and cold-section vessel 7 are physically separate. Due to the separation of the hot section and the cold section, at least a portion of the heat pipes 10 between the hot-section vessel 3 (hot box) and the cold-section vessel 7 (cold box) may be insulated.

In operation, the temperature differential between the hot-section vessel 3 and the cold-section vessel 7 may be at least 200° C.

The cold end 5 of the heat pipes 10 may be submerged in the cooling medium 6 (e.g., boiler feed water or demineralized water). The cooling medium 6 may not completely fill the vessel 7. The arrow 8 indicates the cooling medium 6 entering the cold-section vessel 7. The arrow 9 indicates the cooling medium 6 exiting the cold-section vessel 7, e.g., as water vapor (steam).

The exact arrangement of the heat pipes 10 within the hot-section vessel 3 or cold-section vessel 7 allows for many arrangements. The flow of fluid in the hot-section vessel 3 can be across or normal to the heat pipes 10, along or parallel the heat pipes 10, or a combination. The cold-section vessel 7 can have numerous entry and exit configurations. The heat pipes 10 hot end portion 4 and cold end portion 5 could have fins on their exterior surface to improve heat transfer to the heat pipe. The section of the heat pipe between the hot-section vessel 3 and cold-section vessel 7 can be straight or have bends or twists (helical) to allow for thermal expansion of the heat pipes 10 and vessels 3, 7. In FIG. 7, the cold-section vessel 7 is shown directly above the hot-section vessel 3. However, the heat pipes can be bent such that the two vessels are offset from each other. Moreover, the vessels 3, 7 may be side-by-side at similar elevation.

The working fluid in the heat pipe 10 should vaporize at a temperature at least 50° C. below (or at least 80° C. below) the minimum anticipated temperature of the entering effluent 108. The working fluid in the heat pipe 10 should condense at a temperature at least 25° C. in some instances at least 50° C., above the maximum anticipated temperature of the cooling medium 6. The temperature of the cooling medium 6 should be below the condensation temperature of the working fluid in the heat pipe.

The heat pipes 10 may each have an outer diameter from 1 cm (0.5 inches) to 10 cm (4 inches) and a length up to 10 meters. The heat pipes 10 on the ends may have surface modification, such as fins, ribs, protuberances, pins, or any combination thereof. In some embodiments, the inner surface of the heat pipe is scored with capillary striations to transport the condensed liquid back to the hot end of the heat pipe.

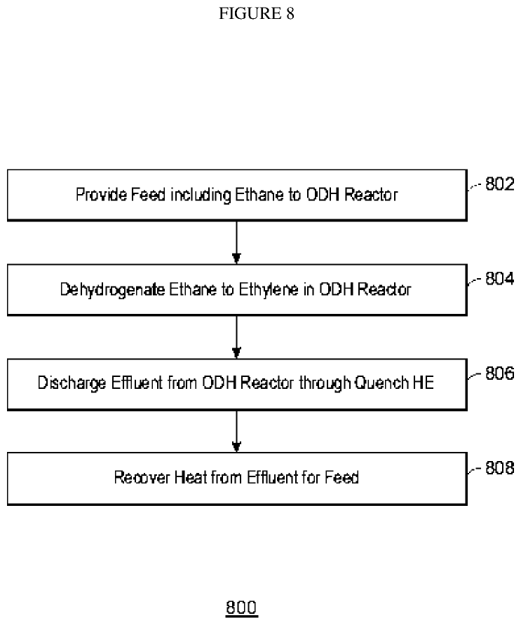

FIG. 8 is a method 800 of operating an ODH reactor system. The ODH reactor system may include at least an ODH reactor and a quench heat exchanger.

At block 802, the method includes feeding ethane, oxygen, and diluent to the ODH reactor having ODH catalyst. The diluent may be, for example, water such as in the form of steam.

At block 804, the method includes dehydrogenating ethane to ethylene via the ODH catalyst in presence of the oxygen in the ODH reactor. Acetic acid may also be formed in the ODH reactor.

At block 806, the method includes discharging effluent from the ODH reactor through the quench heat exchanger, thereby cooling the effluent via the quench heat exchanger. The effluent may include at least ethylene, acetic acid, water, carbon dioxide, carbon monoxide, and unreacted ethane. The residence time of the effluent from the outlet (effluent discharge) of the ODH reactor through the quench heat exchanger to the outlet (effluent discharge) of the quench heat exchanger is less than a specified upper limit. The upper limit may be specified to decrease occurrence of at least one unwanted reaction in the effluent. In certain implementations, the specified upper limit is less than 40 seconds, less than 20 seconds, or less than 10 seconds. In some implementations, the specified upper limit of the residence time is 9 seconds or less.

In implementations, the method may include conveying, via a conduit, the effluent from a discharge of the ODH reactor to the quench heat exchanger. The conduit may include an internal (e.g., static internal) that decreases residence time of the effluent in the conduit. The internal may reduce cross-sectional flow area of a portion of length of the conduit, thereby decreasing the residence time of the effluent in the conduit, and wherein the internal comprises a static internal.

In some implementations, the quench heat exchanger is attached directly to the ODH reactor. The attachment of the quench heat exchanger directly to the ODH reactor may be a flange-to-flange connection involving a flange of an outlet nozzle of the ODH reactor bolted to a flange of an inlet nozzle of the quench heat exchanger. The outlet nozzle or the inlet nozzle, or both, may have an internal, thereby decreasing residence time of the effluent through the outlet nozzle or the inlet nozzle, or both.

In certain implementations, at least a portion of the quench heat exchanger is disposed in the ODH reactor, wherein the effluent discharge outlet of the ODH reactor can be characterized as (or as encompassing) the effluent discharge outlet of the quench heat exchanger. Thus, in those implementations, the residence time of the effluent through the quench heat exchanger can be characterized as zero. In other words, the cooling of the effluent involves cooling the effluent via the quench heat exchanger prior to discharging the effluent from the ODH reactor.

The method may include flowing a cooling medium through the quench heat exchanger to cool the effluent via the quench heat exchanger, wherein the quench heat exchanger is a shell-and-tube heat exchanger. The cooling medium may be water, such as demineralized water, boiler feedwater, or steam condensate. In particular implementations, the method includes generating steam from the cooling medium via the shell-and-tube heat exchanger with heat from the effluent. Steam can also be generated with the quench heat exchanger as a heat-pipe heat exchanger and the cooling medium as water.

At block 808, the method may include flowing the effluent from the quench heat exchanger through a feed heat exchanger (a cross exchanger) to heat the feed with the effluent. Thus, the effluent may be further cooled.

The Examples below demonstrate that unwanted reactions occur in mixtures similar to the ODH reactor effluent 108 (FIGS. 1-3). Such unwanted reactions increasingly occur at increasing temperature. For instance, in the Examples, the presence or extent of the unwanted reactions was more at 350° C. compared to 250° C.

As discussed, the temperature of the effluent 108 as discharged from the ODH reactor 102 can be, for example, in the range of 300° C. to 450° C. The Examples generally support that the presence or extent of the unwanted reactions are greater in this temperature range than if cooled to below this temperature range. Further, the Examples and basic chemical principles support that the more time this mixture is at the elevated temperature in that temperature range of 300° C. to 450° C., the greater the extent of the unwanted reactions. The Examples indicate that limiting exposure of the mixture to the elevated temperature to less than one minute can be beneficial to reduce the extent of the unwanted reactions.

A residence time that may be of interest is the length of time that the ODH reactor effluent 108 is above an upper temperature threshold (e.g., about 225° C., 250° C., 275° C., or 300° C.), which is from the ODH reactor 102 outlet to some point in the effluent 108 flow path through the quench heat exchanger 104. Implementations conservatively specify the process (effluent) outlet of the quench heat exchanger 104 as the point at which the effluent 108 is cooled to below the specified upper temperature threshold. In the Examples below, the lab configuration was employed as a heater and a volume for residence time. The reactor in the Examples did not have ODH catalyst. Therefore, the reactor in the Examples was not utilized as a typical ODH reactor. In the Examples, mixtures resembling typical ODH reactor effluent were fed through the lab preheater and reactor (no ODH catalyst). The lab setup was used to evaluate typical ODH reactor effluent at various temperatures and residence time at those temperatures. The Examples generally do not give an exact number for residence time to specify for any commercial implementation. Instead, the Examples give two basic related conclusions: (1) recognition of the problem of undesired reactions in ODH reactor effluent at above about 250° C.; and (2) a ballpark (order of magnitude, e.g., less than 1 minute) of what the maximum residence time should be for the effluent at above about 250° C. Again, the Examples do not necessarily give precise numerical values (seconds) of residence time as calculated in the laboratory for a commercial implementation. The calculations of residence time in the Examples were not based on actual conditions of pressure and temperature. Even so, as mentioned, the Examples (a) identify that there is a problem of unwanted reactions; and (b) give an understanding (approximation or order of magnitude) of what should be the maximum temperature and maximum residence time of the ODH reactor effluent to avoid significant unwanted reactions.

An embodiment is a method of operating an ODH reactor system. The method includes feeding ethane, oxygen, and diluent (e.g., water as steam) to an ODH reactor having ODH catalyst. The method includes dehydrogenating ethane to ethylene via the ODH catalyst in presence of the oxygen in the ODH reactor, thereby forming acetic acid in the ODH reactor. The method includes discharging effluent from the ODH reactor through a quench heat exchanger, thereby cooling the effluent via the quench heat exchanger to below a temperature threshold (e.g., in a range of 200° C. to 300° C.). The method may include specifying the temperature threshold at a value less than 275° C. (or less than 250° C.) and above a dew point of the effluent. The effluent includes ethylene, acetic acid, water, carbon dioxide, carbon monoxide, and unreacted ethane. The residence time of the effluent from the ODH reactor to an outlet of the quench heat exchanger that discharges the effluent is less than a specified upper limit. The specified upper limit may be, for example, a value less than 60 seconds or a value less than 20 seconds. The specified upper limit may be specified to decrease occurrence of an unwanted reaction in the effluent. The method may include conveying, via a conduit, the effluent from an outlet (effluent discharge outlet) of the ODH reactor to the quench heat exchanger. The conduit may have an internal (e.g., a static internal) that decreases residence time of the effluent in the conduit. The internal (e.g., a static mixer) may reduce volume of the conduit available for flow of the effluent, thereby decreasing the residence time of the effluent in the conduit. In implementations, the quench heat exchanger may be attached directly to the ODH reactor. The attachment of the quench heat exchanger directly to the ODH reactor may involve a flange-to-flange connection that is a flange of an outlet nozzle of the ODH reactor bolted to a flange of an inlet nozzle of the quench heat exchanger. The outlet nozzle or the inlet nozzle, or both, may have an internal, thereby decreasing residence time of the effluent through the outlet nozzle or the inlet nozzle, or both. In implementations, at least a portion of the quench heat exchanger may be disposed in the ODH reactor, wherein discharge of the ODH reactor includes the discharge of the quench heat exchanger, wherein the residence time is zero. In these implementations, the cooling of the effluent may be cooling the effluent via the quench heat exchanger prior to discharging the effluent from the ODH reactor.

Another embodiment is a method of an ODH reactor system, the method including providing feed including ethane and oxygen to an ODH reactor, and dehydrogenating ethane to ethylene via ODH catalyst in the ODH reactor. The method includes discharging effluent from the ODH reactor through a quench heat exchanger, thereby cooling the effluent via the quench heat exchanger to below a specified temperature threshold, such as a value less than 300° C. (or values less than 250° C.) and that is above the dew point of the effluent. The temperature threshold may be, for example, in the range of 200° C. to 300° C. The effluent includes ethylene, acetic acid, water, carbon dioxide, carbon monoxide, and unreacted ethane. Residence time of the effluent from an outlet of the ODH reactor that discharges the effluent to an outlet of the quench heat exchanger that discharges the effluent as cooled is less than an upper limit (e.g., a value less than 40 seconds or a value less than 60 seconds) as specified to decrease occurrence of an unwanted reaction in the effluent. The method may include flowing a cooling medium through the quench heat exchanger to cool the effluent via the quench heat exchanger, wherein the quench heat exchanger is a shell-and-tube heat exchanger. The cooling medium may include water, such as demineralized water, boiler feedwater, or steam condensate. The method may include generating steam from the cooling medium via the shell-and-tube heat exchanger with heat from the effluent. In implementations, the quench heat exchanger may be a heat-pipe heat exchanger. Lastly, the method may include flowing the effluent from the quench heat exchanger through a feed heat exchanger that is a cross exchanger to heat the feed with the effluent, thereby further cooling the effluent.

Yet another embodiment is an ODH reactor system including an ODH reactor having ODH catalyst to dehydrogenate ethane in presence of oxygen to ethylene and generate acetic acid. The ODH reactor system includes a quench heat exchanger to cool effluent of the ODH reactor to below a threshold temperature (e.g., a value less than 300° C.). The effluent includes ethylene, acetic acid, water, carbon dioxide, carbon monoxide, and unreacted ethane. The ODH reactor system is configured to provide residence time of the effluent from an effluent outlet of the ODH reactor to an effluent outlet of the quench heat exchanger less than an upper limit (e.g., a value less than 60 seconds) as specified to decrease occurrence of an unwanted reaction in the effluent. The ODH reactor system may include a conduit to convey the effluent from the outlet of the ODH reactor to the quench heat exchanger. The conduit may include a static internal disposed in the conduit to reduce flow volume of the conduit to decrease residence time of the effluent in the conduit. The quench heat exchanger may be a shell-and-tube heat exchanger configured to receive a cooling medium to cool the effluent. The shell-and-tube heat exchanger may be configured to receive boiler feedwater as the cooling medium to facilitate generation of steam from the boiler feedwater with heat from the effluent. In implementations, the quench heat exchanger may be a heat-pipe heat exchanger. In implementations, the quench heat exchanger may be directly attached to the ODH reactor. The quench heat exchanger may be directly attached to the ODH reactor via a flange-to-flange connection in which a flange of an outlet nozzle of the outlet of the ODH reactor is bolted to a flange of an inlet nozzle of the quench heat exchanger. A static internal may be disposed in the outlet nozzle or the inlet nozzle, or both, to decrease residence time of the effluent through the outlet nozzle or the inlet nozzle, or both. Lastly, at least a portion of the quench heat exchanger may disposed in the ODH reactor to give the residence time as zero, and wherein to cool the effluent involves to cool the effluent prior to discharge of the effluent from the ODH reactor.

### EXAMPLES

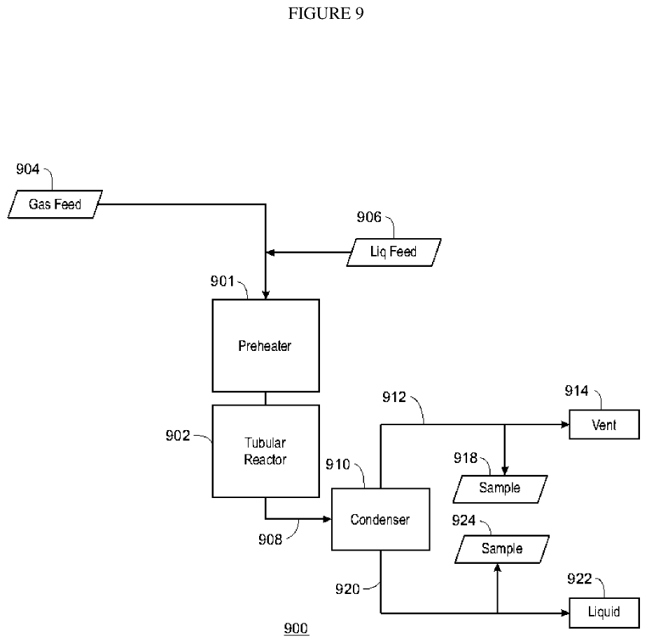

The Examples are given only as examples and not intended to limit the present techniques. Examples 1-5 are presented. Examples 1-5 were performed in a laboratory reactor system (FIG. 9) approaching pilot scale. In the Examples, mixtures resembling ODH reactor effluent were fed through the lab preheater and lab reactor to the lab condenser. The reactor in the Examples was not used to resemble the ODH reactor (there was no ODH catalyst in the lab reactor in the Examples). Instead, the reactor in the Examples was merely a wide spot in the line and used as a heater to maintain the mixture at a specified temperature. The Examples were directed to identifying unwanted reactions in typical ODH reactor effluent, and at what temperatures and how much time needed for those undesired reactions to occur.

FIG. 9 is the laboratory reactor system 900 utilized to perform the Examples 1-5. The system 900 includes a tubular preheater 901 (a steam generator) and a tubular reactor 902 disposed downstream of the tubular preheater 901. ODH catalyst was removed from the tubular reactor 902, such that no ODH catalyst was disposed in the tubular reactor 902. The tube of the tubular reactor 902 was constructed of Type 316L stainless steel. The tube of the tubular preheater 901 was constructed of Hastelloy C-276. The tubular reactor 902 had a heat transfer jacket that received circulating oil from a closed-loop oil bath for heating or cooling of the contents in the tube of the tubular reactor 902 to maintain a desired temperature in the reactor 902. The preheater 901 was equipped with electrical mantel heating placed in contact with the preheater 901 tube via case aluminum jacket to heat the contents in the tube of the preheater 901.

The tube of the preheater 901 had an inside diameter of 0.94 centimeter (cm), a height of 381 cm, and an inside volume of 381 cubic centimeters (cm³). The tube of the tubular reactor 902 had an inside diameter of 2.12 cm, a height of 170 cm, and an inside volume of 599 cm³. The temperature of the tubular reactor 902 was monitored with thermocouples as temperature sensors. Again, the tubular reactor 902 was not loaded with catalyst, and thus served as a volume for residence time and served as a heater to give temperature in which unwanted reactions may occur.

A combined gas feed 904 was fed to the inlet of the preheater 901 from respective gas cylinders. The gas components in the gas feed 904 included combinations of ethylene, oxygen gas, or ethane. The gas cylinders were obtained from Praxair, Inc. having headquarters in Danbury, Connecticut, USA. The available pressure of the gas cylinders provided motive force for flow of the combined gas feed 904 into the preheater 901. A respective mass flow controller (operating at 21° C.) associated with each gas cylinder gave the desired flow rate of each gas component. A liquid feed 906 (combined liquid feed) was introduced into the gas feed 904 flowing to the preheater 901. The liquid feed 906 included water and acetic acid. In Example 5, ethanol was added to the liquid feed. A mass flow controller (operating at 21° C.) controlled the flow rate of the liquid feed 906. The liquid feed 906 evaporated in the preheater 901.

The inlet pressure (psig) at the reactor 902 inlet was measured via a pressure sensor. This reactor 902 inlet pressure was due to the hydraulic backpressure generated by flow of the feed gas through the reactor 902, downstream condenser 910, and associated piping, as well as hydraulic backpressure provided by a back pressure regulator located downstream of the condenser 910.

The discharge stream 908 (labeled as product in the Examples) discharged from the reactor 902 to the condenser 910 (partial condenser), which condensed components of the vaporized liquid feed 906 in the reactor discharge stream 908. The cooling medium in the condenser 910 was distilled water. The condenser 910 was a shell-and-tube heat exchanger with operation of the reactor discharge stream 908 on the tube side and the distilled water on the shell side. Product gas 912 discharged from the condenser 910 to a vent system 914. A sample syringe was utilized to collect a gas sample 918 of the product gas 912 at a sample point downstream of the condenser 910. Liquid product 920 discharged from the condenser 910 to a liquid collection system 922. A liquid sample 924 of the liquid product 920 was obtained. The gas samples 918 and the liquid samples 924 were analyzed for composition via a gas chromatograph.

The residence time considered in the Examples was the combined residence time of the residence time of the evaluated mixture in the preheater 901 plus the residence time of the evaluated mixture in the reactor 902. The residence time of the mixture through the small tubing between the preheater 901 and the reactor 902 was negligible. The residence time of the mixture through the small tubing between the reactor 902 and the condenser 910 was negligible. The residence time of the mixture through the condenser 910 was negligible. In contrast, the residence time of lengthy conduits in commercial-scale implementations and through condensers (heat exchangers) of larger size (commercial scale) can be relatively significant.

The residence time of the mixture in the preheater 901 and the residence time in the reactor 902 were calculated based on pressure specified at 1 atmosphere (atm) absolute and temperature specified at 21° C., which was the temperature of the gas feed 904 and the liquid feed 906. The liquid feed 906, while a liquid at 21° C., was arbitrarily specified as vapor at 22.4 liters per mole in the preheater 901 and in the reactor 902. The actual pressure as measured at the inlet of the reactor 902 was 61 psig or 62 psig. The actual temperature in the reactor 902 was 250° C. or greater. Thus, the residence time in the Examples was not based on actual conditions of pressure and temperature. Therefore, the residence time as calculated in the Examples is not true residence time but instead an order-of-magnitude approximation for residence time that may be applicable to commercial implementations for limiting the time of the ODH reactor effluent at elevated temperature. Lastly, as noted in Examples 1-5 below, the combined residence time (as calculated) of the evaluated mixture in the preheater 901 (3 seconds) plus the residence time of the evaluated mixture in the reactor 902 (6 seconds) is 9 seconds. Because of the calculation technique implemented in the Examples, the volumetric flow rate as approximated was identical for all Examples 1-5. In other words, the volumetric flow rate was not affected by differences in composition of the evaluated mixtures among Examples 1-5. Thus, the residence time as calculated was the same 9 seconds for all Examples 1-5.

In Examples 1-5, the formation of CO, CO₂, and oxygenated solid fouling was likely due to combination of gas phase reactions and surface catalytic reaction over the interior surface of the reactor 902 tube.

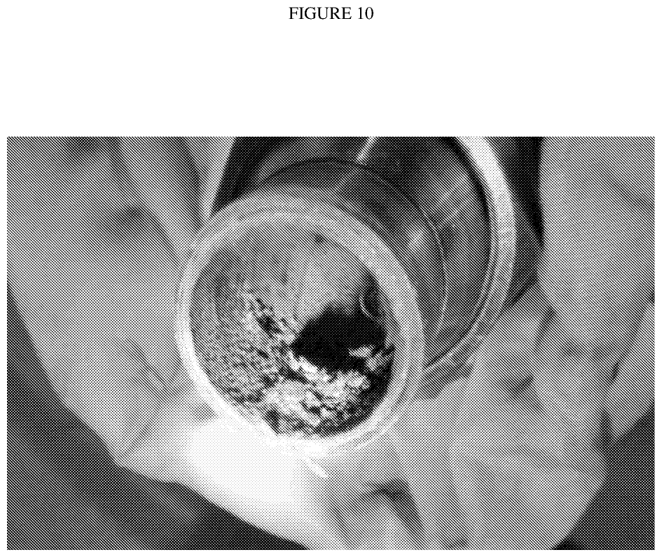

After the Examples 1-5 were completed, the reactor 902 was opened and inspected. Approximately 2 grams of a fouling material (see FIG. 10) was observed. This approximate 2 grams of the fouling material was formed in the Examples 1-5 collectively. FIG. 10 depicts a sample of the fouling material in the bottom (lower) portion of the reactor 902 prior to collection of the sample. The results of the elemental carbon-hydrogen-nitrogen-oxygen (CHNO) analysis for the sample of this material are shown in Table 1 below. Based on this CHNO analysis result, it can be inferred that 28.4 wt. % of the sample are organic elements while the remainder are inorganic elements. The dominant organic elements were found to be oxygen element and carbon. In operation, for cases that % drop in O₂ dry mole fraction does not match the % increase (or % generated) undesirable oxygenated byproducts (such as CO, CO₂, acetic acid), it is speculated that O₂ formed as elemental oxygen in this collected solid fouling material.

TABLE 1
CHNO Results for Fouling Sample Collected
from Laboratory Tubular Reactor

| | | | | |
|---|---|---|---|---|
| C | H | N | O | Total |

Mass Fraction (wt %)

| | | | | |
|---|---|---|---|---|
| 9.92 | 0.58 | <0.04 | 17.90 | 28.44 |

Mole Fraction (mol % - normalized to 100%)

| | | | | |
|---|---|---|---|---|
| 32.77 | 22.90 | <0.12 | 44.22 | 100.00 |

The five most prevalent inorganic elements of the sample as determined by ICP-MS analysis were sodium (Na) (8.0 wt. %), aluminum (Al) (5.0 wt. %), Te (3.2 wt. %), Mo (2.4%), and iron (Fe) (2.2 wt. %). A source of Al and Te may have been trace residue of ODH catalyst (and catalyst support) on the inner surface of reactor 902 tube from previous routine experiments utilizing ODH catalyst. Similarly, a source of Mo may have been residue of catalyst active phase on the inner surface of reactor 902 tube from previous routine experiments. A source of Fe and Mo could have been corrosion of this reactor 902 tube and preheater 901 tube, which are constructed of stainless steel 316 and Hastelloy C-276, respectively. A source of Na may have been collectively from (1) feed water-oxygenate liquid mixture injected to the reactor 902, (2) Na impurity in the residue of alumina catalyst support (from previous experiments), and (3) external impurity introduced to the mixture during mixture handling and preparation.

In Examples, an increase in ethylene dry-gas volume fraction was observed. It was assumed that the increase in ethylene dry-gas volume fraction is an artifact of consuming more O₂ compared to ethylene on molar basis. This implied that the increase in dry-gas volume fraction of ethylene is not reflective of increase in volume flow rate of this compound in the product stream. The consumption of ethylene and O₂ was attributed to formation of undesirable byproducts (mainly CO and CO₂) and the aforementioned solid fouling. Conversion of ethylene and O₂ to the mentioned undesirable byproducts can be explained, for example, based on the two bulk reactions [1] C₂H₄+3 O₂→2 CO₂+2 H₂O and [2] C₂H₄+2 O₂→2 CO+2 H₂O. This confirmed higher relative consumption of O₂ compared to ethylene on molar basis. The CHNO analysis conducted on the solid fouling also confirmed higher relative consumption of O₂ compared to ethylene on molar basis.

For a commercial-scale ODH reactor that dehydrogenates ethane to ethylene via ODH catalyst in presence of oxygen, reactions in the effluent from the ODH reactor were considered in the laboratory system in the Examples. Note that reference to a commercial-scale ODH reactor refers to a hypothetical commercial-scale reactor and not to an actual implementation of a commercial-scale reactor.

In order to explore and mimic the presence of reactions in the effluent from an ODH reactor, respective mixtures resembling ODH reactor effluent were fed through the preheater (steam generator) and tubular reactor (no catalyst) in the laboratory system in Examples 1-5. The mixtures were labeled as feed. Again, the total residence time through the preheater 901 and the tubular reactor 902 combined may be considered for comparison on an order-of-magnitude estimation basis to the residence time in the field from the ODH reactor outlet (for effluent discharge) to the outlet of the quench heat exchanger (for effluent discharge). Reactions of interest included any gas phase reactions and any reactions catalyzed by the inside metal surface of the reactor 902 vessel tube. The pilot tubular reactor 902 had no catalyst but provided temperature control of the contents. The discharge from the tubular reactor 902 was labeled as product and flowed through a partial condenser 910 (heat exchanger) that discharged a product gas 912 stream and a product liquid 920 stream.

The residence times given in the Tables below for all Examples 1-5 have an identical basis. The basis is a reactor inside volume of 599 cm³, a preheater inside volume is 381 cm³, and total feed flow rate of 3873 cm³/min at 1 atm absolute and 21° C. with the liquid feed considered as vapor at 22.4 liters per mole of liquid. The liquid components as considered vaporized contribute to the 3873 cm³/min.

### Example 1

The mixture as feed in Example 1 through the preheater 901 and reactor 902 included water, acetic acid, ethylene, and oxygen. The ethylene and oxygen were gas. The water and acetic acid were liquid in the feed mixture but vaporized in the preheater. The feed composition and operating conditions are reported in Table 2. The dry gas 904 composition of the feed and the dry gas composition of the product gas 912 (from the partial condenser) are reported in Table 3. The liquid 904 composition of the feed and the liquid composition of the product liquid (from the partial condenser) are reported in Table 4. In considering the experimental results in Example 1, the following observations were made with the tubular reactor 902 at an operating temperature of 250° C. and with total residence time (preheater and tubular reactor combined) of 9 seconds as calculated.

Ethane dry gas volume fraction increased (from 0%) in the product stream compared to feed stream (0.04% absolute increase).

Ethylene dry gas volume fraction increased in the product stream compared to feed stream (0.72% absolute increase). This increase was assumed not to be representative of real increase in volumetric flow rate of ethylene due to being an artifact of O₂/ethylene conversion to undesirable byproducts and solid fouling as explained above.

Oxygen dry gas volume fraction decreased in the product stream compared to feed stream (0.79% absolute decrease)

Acetic acid liquid mass fraction decreased in the product stream compared to feed stream (1.46% absolute decrease)

From the observed decrease in O₂ dry volume fraction, decrease in acetic acid liquid mass fraction, formation of oxygenated solid fouling, and formation of trace amount of ethane, it can be inferred that at a temperature of 250° C., detectable thermal reaction occurred. The thermal reaction included formation of solid fouling and trace amount of ethane. This suggests that is beneficial this feed mixture be rapidly cooled down at operating temperature below about 250° C. or 275° C., and the residence time at elevated temperature be less than one minute based on the order-of-magnitude estimate in the laboratory of about 9 seconds. Such may avoid the loss of ethylene, O₂, and acetic acid product mixture to undesirable solid fouling and conversion of trace amounts to ethane.

TABLE 2
Example 1. Operating Conditions and
Feed Composition for Examples 1-5

| Residence Time (s) | |
|---|---|
| Preheater | 3 |
| Reactor | 6 |
| Temperature (° C.) | |
| Preheater | 247 |
| Reactor | 250 |
| Reactor Inlet Pressure (psig) | 62 |
| Feed Composition (vol. %) | |
| H₂0 | 70 |
| CH₃COOH | 2 |
| C₂H₆ | 0 |
| C₂H₅OH | 0 |
| C₂H₄ | 23 |
| O₂ | 5 |
| CO₂ | 0 |

TABLE 3
Example 1. Dry Gas Composition of Feed and Product

| | Dry Gas Composition (vol. %) | | | | | | |
|---|---|---|---|---|---|---|---|
| | C₂H₂ | C₂H₆ | C₂H₄ | CO | CO₂ | CH₄ | O₂ |
| Feed | 0.00 | 0.00 | 82.14 | 0.00 | 0.00 | 0.00 | 17.86 |
| Product | 0.00 | 0.04 | 82.86 | 0.00 | 0.00 | 0.00 | 17.06 |

Note:
the average of data for two experiments were used.

TABLE 4
Example 1. Liquid Composition of Feed and Product

| | Liquid Composition (wt. %) | | | | | |
|---|---|---|---|---|---|---|
| | | | | Acetic | | Other |
| | H₂O | Methanol | Ethanol | Acid | Acetone | Oxygenates |
| Feed | 90.52 | 0 | 0 | 9.48 | 0 | 0 |
| Product | 91.96 | 0 | 0 | 8.02 | 0 | 0 |

Note:
the average of data for two experiments were used.

### Example 2

The mixture fed through the preheater and tubular reactor (no catalyst) in Example 2 included water, acetic acid, ethylene, and oxygen. Example 2 was evaluated at 350° C. compared to Example 1 evaluated at 250° C. The feed composition and operating conditions are reported in Table 5. The dry feed gas and product gas compositions are reported in Table 6. The liquid feed and product compositions are reported in Table 7. In consideration of the experimental results, the following observations were made at total calculated residence time of 9 seconds and temperature of 350° C.

Ethane dry gas volume fraction increased (from 0%) in the product stream compared to feed stream (0.05% absolute increase).

Ethylene dry gas volume fraction decreased in the product stream compared to feed stream (1.63% absolute decrease).

Oxygen dry gas volume fraction increased in the product stream compared to feed stream (0.71% absolute increase). The increase in O₂ dry volume gas fraction has not been observed for any the other Examples 1 and 3-4), therefore it is speculated that this increase likely due to some minor GC analysis error.

CO dry gas volume fraction increase (from 0%) in the product stream compared to feed stream (0.47% absolute increase).

CO₂ dry gas volume fraction increased (from 0%) in the product stream compared to feed stream (0.41% absolute increase).

Acetic acid liquid mass fraction increased in the product stream compared to feed stream (1.18% absolute increase)

From the observed decrease in ethylene dry gas volume fraction, increase in acetic acid liquid mass fraction, formation of oxygenated solid fouling, formation of COₓ (CO and CO₂), and formation of trace amount of ethane, it can be inferred that at the loosely approximated combined residence time of 9 seconds and the reactor temperature of 350° C., detectable thermal reaction occurred (leading into formation of solid fouling, acetic acid, CO, CO₂ and trace amount of ethane). This may suggest this that the mixture should be rapidly cooled down at operating temperature below 350° C. and at residence time at elevated temperature less than one minute to avoid the loss of ethylene (and O₂) acetic acid, CO, CO₂, trace amount of ethane, and to undesirable solid fouling.

Comparing the result of Example 2 to Example 1, for a lower reactor temperature of 250° C. (compared to 350° C.), the rate of the unwanted thermal reactions was decreased noticeably as evidenced by no generation of CO and no generation of CO₂ in the product stream of the experiment conducted at reactor temperature of 250° C. Note in these two comparative experiments of Examples 1 and 2, the reactor operating conditions (except for reactor temperature) and the feed composition were the same to facilitate studying the effect of reactor temperature on rate of the mentioned unwanted thermal reactions. The reaction(s) responsible for forming solid oxygenated compound may be a function of the feed adsorbing or chemisorbing on the tube metal inner surface that acts as catalyst. In general, the rate of adsorption/chemisorption increases as the temperature decreases. Increasing temperature could lead to formation of a greater amount of solid oxygenated fouling.

TABLE 5
Example 2. Operating Conditions and Feed Composition

| Residence Time (s) | |
|---|---|
| Preheater | 3 |
| Reactor | 6 |
| Temperature (° C.) | |
| Preheater | 247 |
| Reactor | 350 |
| Reactor Inlet Pressure (psig) | 62 |
| Feed Composition (vol. %) | |
| H₂0 | 70 |
| CH₃COOH | 2 |
| C₂H₆ | 0 |
| C₂H₄ | 23 |
| O₂ | 5 |
| CO₂ | 0 |

TABLE 6
Example 2. Dry Gas Composition of Feed and Product

| | Dry Gas Composition (vol %) | | | | | | |
|---|---|---|---|---|---|---|---|
| | C₂H₂ | C₂H₆ | C₂H₄ | CO | CO₂ | CH₄ | O₂ |
| Feed | 0.00 | 0.00 | 82.14 | 0.00 | 0.00 | 0.00 | 17.86 |
| Product | 0.00 | 0.05 | 80.51 | 0.47 | 0.41 | 0.00 | 18.57 |

Note:
the average of data for two experiments were used.

TABLE 7
Example 2. Liquid Composition of Feed and Product

| | Liquid Composition (wt. %) | | | | | |
|---|---|---|---|---|---|---|
| | | | | Acetic | | Other |
| | H₂O | Methanol | Ethanol | Acid | Acetone | Oxygenates |
| Feed | 89.94 | 0 | 0 | 10.06 | 0 | 0 |
| Product | 88.76 | 0 | 0 | 11.24 | 0 | 0 |

Note:
the average of data for two experiments were used.

### Example 3

The mixture fed in Example 3 through the preheater (steam generator) and tubular reactor (no catalyst) included water, acetic acid, and ethylene. The feed composition and operating conditions are reported in Table 8. The dry feed gas and product gas compositions are reported in Table 9. The liquid feed and product compositions are reported in Table 10. In considering the experimental results, the following observations were made at temperature of 350° C.

Ethane dry gas volume fraction increased (from 0%) in the product stream compared to feed stream (0.08% absolute increase).

Ethylene dry gas volume fraction decreased in the product stream compared to feed stream (0.41% absolute decrease).

No oxygen was present in the feed or product of Example 3.

CO₂ dry gas volume fraction increased (from 0%) in the product stream compared to feed stream (0.05% absolute increase).

Acetic acid liquid mass fraction decreased in the product stream compared to feed stream (3.52% absolute decrease).

Based on the observed decrease in ethylene dry-gas volume fraction, decrease in acetic-acid liquid mass fraction, formation of oxygenated solid fouling, formation of CO₂, and formation of trace amount of ethane, it can be inferred that at the temperature of 350° C., detectable thermal reaction occurred. This suggests that this mixture should be rapidly cooled down to an operating temperature below 350° C. to avoid the conversion of ethylene, O₂, and acetic acid to undesirable solid fouling, CO₂, and trace amount of ethane.

With the absence of O₂ in the feed in Example 3 (compared to presence of O₂ in the feed stream in Example 2), the rate of the unwanted thermal reactions towards COₓ (CO and CO₂) was decreased and towards acetic acid was suppressed as evidenced by no generation of CO, decrease in CO₂ volume fraction, and decrease in acetic acid weight fraction in the product. However, because acetic acid was present in the feed, the decrease in weight fraction of acetic acid may be due to conversion of acetic acid into the oxygenated fouling. The decrease in consumption of acetic acid may correspond into an increase in the rate of formation of solid oxygenated fouling from the acetic acid. In both cases of presence of O₂ and absence of O₂, unwanted reactions are present leading into different distribution of unwanted reactions giving unwanted byproducts and solid fouling. In these two comparative experiments of Examples 2 and 3, the reactor operating conditions and relative feed composition of H₂O/acetic acid/ethylene remained unchanged to facilitate studying the effect of presence of feed O₂ on rate of the mentioned unwanted thermal reactions.

TABLE 8
Example 3. Operating Conditions and Feed Composition

| Residence Time (s) | |
|---|---|
| Preheater | 3 |
| Reactor | 6 |
| Temperature (° C.) | |
| Preheater | 247 |
| Reactor | 250 |
| Reactor Inlet Pressure (psig) | 61 |
| Feed Composition (vol. %) | |
| H₂0 | 74 |
| CH₃COOH | 2 |
| C₂H₆ | 0 |
| C₂H₄ | 24 |
| O₂ | 0 |
| CO₂ | 0 |

TABLE 9
Example 3. Dry Gas Composition of Feed and Product

| | Dry Gas Composition (vol. %) | | | | | | |
|---|---|---|---|---|---|---|---|
| | C₂H₂ | C₂H₆ | C₂H₄ | CO | CO₂ | CH₄ | O₂ |
| Feed | 0.00 | 0.00 | 100.00 | 0.00 | 0.00 | 0.00 | 0.00 |
| Product | 0.00 | 0.08 | 99.59 | 0.00 | 0.05 | 0.00 | 0.00 |

Note:
the average of data for two experiments were used.

TABLE 10
Example 3. Liquid Composition of Feed and Product

| | Liquid Composition (wt. %) | | | | | |
|---|---|---|---|---|---|---|
| | | | | Acetic | | Other |
| | H₂O | Methanol | Ethanol | Acid | Acetone | Oxygenates |
| Feed | 87.59 | 0 | 0 | 12.41 | 0 | 0 |
| Product | 91.11 | 0 | 0 | 8.69 | 0 | 0 |

Note:
the average of data for two experiments were used.

### Example 4

The mixture fed through the preheater and tubular reactor (no catalyst) in Example 4 included water, carbon dioxide, acetic acid, ethane, and oxygen. Ethane was used in place of ethylene in the feed. The feed composition and operating conditions are reported in Table 11. The dry feed gas and product gas compositions are reported in Table 12. The liquid feed and product compositions are reported in Table 13. In considering the experimental results, the following observations were made at temperature of 350° C.

Ethane dry gas volume fraction decreased in the product stream compared to feed stream (0.24% absolute decrease).

Oxygen dry gas volume fraction decreased in the product stream compared to feed stream (0.94% absolute decrease)

CO₂ dry gas volume fraction increased in the product stream compared to feed stream (1.17% absolute increase).

Acetic acid liquid mass fraction decreased in the product stream compared to feed stream (9.93% absolute decrease).

Based on the observed decrease in ethane dry gas volume fraction, decrease in acetic-acid liquid mass fraction, decrease in oxygen dry gas volume fraction, increase in CO₂ dry gas volume fraction, formation of oxygenated solid fouling, it can be inferred that detectable undesirable thermal reactions occurred at 350° C. Such suggests that this mixture this should be rapidly cooled down to an operating temperature below 350° C.

TABLE 11
Example 4. Operating Conditions and Feed Composition

| Residence Time (s) | |
|---|---|
| Preheater | 3 |
| Reactor | 6 |
| Temperature (° C.) | |
| Preheater | 247 |
| Reactor | 350 |
| Reactor Inlet Pressure (psig) | 62 |
| Feed Composition (vol. %) | |
| H₂0 | 7 |
| CH₃COOH | 2 |
| C₂H₆ | 16 |
| C₂H₄ | 0 |
| O₂ | 0 |
| CO₂ | 70 |

TABLE 12
Example 4. Dry Gas Composition of Feed and Product

| | Dry Gas Composition (vol. %) | | | | | | |
|---|---|---|---|---|---|---|---|
| | C₂H₂ | C₂H₆ | C₂H₄ | CO | CO₂ | CH₄ | O₂ |
| Feed | 0.00 | 17.58 | 0.00 | 0.00 | 76.92 | 0.00 | 5.49 |
| Product | 0.00 | 17.35 | 0.00 | 0.00 | 78.40 | 0.00 | 4.56 |

Note:
the average of data for two experiments were used.

TABLE 13
Example 4. Liquid Composition of Feed and Product

| | Liquid Composition (wt. %) | | | | | |
|---|---|---|---|---|---|---|
| | | | | Acetic | | Other |
| | H₂O | Methanol | Ethanol | acid | Acetone | Oxygenates |
| Feed | 46.32 | 0 | 0 | 53.68 | 0 | 0 |
| Product | 56.25 | 0 | 0 | 43.75 | 0 | 0 |

Note:
the average of data for two experiments were used.

### Example 5

The mixture fed through the preheater and reactor (no catalyst) included water, acetic acid, ethanol, ethylene, and oxygen. The feed composition and operating conditions are reported in Table 14. The presence of ethanol (C₂H₅OH) in this feed mixture is meant to mimic the presence of this compound due to one or both of the following reasons: (1) external ethanol injection to the last section of an ODH reactor due to process needs, and (2) presence of ethanol as a byproduct or contaminant in the ODH product effluent. The dry feed gas and product gas compositions are reported in Table 15. The liquid feed and product compositions are reported in Table 16. The liquid product sample was only collected from the experiment conducted at reactor temperature of 325° C. (Example 5-a). Experiments at reactor temperatures of 334° C. (Example 5-b) and 340° C. (Example 5-c) were conducted with gas analysis only to screen the effect of temperature increase on dry gas composition and to understand if excessive reaction could occur at operating temperature higher than 325° C. Therefore, no liquid sample was collected or analyzed for these experiments conducted at 334° C. and 340° C. It is noteworthy to mention that at operating temperature of 340° C., shortly after reaching this reaction temperature, the reactor did not remain in steady state and eventually led into excess reaction close to outlet of the reactor. Bearing this detail in mind, looking at the experimental results, the following observations were made at reaction temperature of 325° C.

Ethane dry gas volume fraction increased (from 0%) in the product stream compared to feed stream (0.04% absolute increase).

Ethylene dry gas volume fraction increased in the product stream compared to feed stream (6.20% absolute increase). This increase was assumed not to be representative of real increase in volumetric flow rate of ethylene due to being an artifact of O₂/ethylene conversion to undesirable byproducts and solid fouling as discussed.

CO dry gas volume fraction increased (from 0%) in the product stream compared to feed stream (0.04% absolute increase).

Oxygen dry gas volume fraction decreased in the product stream compared to feed stream (6.41% absolute decrease).

CO₂ dry gas volume fraction increased in the product stream compared to feed stream (0.12% absolute increase).

Acetic acid liquid mass fraction decreased in the product stream compared to feed stream (1.40% absolute decrease).

Ethanol liquid mass fraction decreased in the product stream compared to feed stream (0.25% absolute decrease).

From the observed decrease in O₂ dry gas volume fraction, trace increase in CO dry gas volume fraction, trace increase in CO₂ dry gas volume fraction, trace increase in ethane dry gas volume fraction, decrease in acetic-acid liquid mass fraction, decrease in ethanol liquid mass fraction, and formation of oxygenated solid fouling, it can be inferred that at the temperature of 325° C., detectable undesired thermal reactions occurred. Such suggest that this mixture should to be rapidly cooled down to below an operating temperature of 325° C.

In considering the experimental results, the following observations were made at the temperature of 340° C.

Ethane dry gas volume fraction in the product stream remained almost unchanged as temperature was increased. In all cases trace amount (≤0.05 vol. %) was observed.

CO dry gas volume fraction in the product stream remained almost unchanged as temperature was increased. In all cases trace amount (≤0.04 vol. %) was observed. O₂ dry gas volume fraction in the product stream decreased as temperature was increased.

Ethylene dry gas volume fraction in the product stream increased as temperature was increased. This increase was assumed not to be representative of real increase in volumetric flow rate of ethylene due to being an artifact of O₂/ethylene conversion to undesirable byproducts and solid fouling.

From the observed decrease in O₂ dry gas volume fraction, unchanged dry gas volume fraction of CO, unchanged dry gas volume fraction of ethane, observed excess reaction at highest operating temperature of 340° C. and formation of oxygenated solid fouling, it can be inferred that as reaction temperature was increased from 325° C. to 340° C., the rate of fouling formation increased leading into eventual excessive reaction.

TABLE 14
Example 5. Operating Conditions and Feed
Composition for Three Experiments

| | 5a | 5b | 5c |
|---|---|---|---|
| Residence Time (s) | | | |
| Preheater | 3 | 3 | 3 |
| Reactor | 6 | 6 | 6 |
| Temperature (° C.) | | | |
| Preheater | 247 | 247 | 247 |
| Reactor | 325 | 334 | 340 |
| Reactor Inlet Pressure (psig) | 62 | 62 | 62 |
| Feed Composition (mol. %) | | | |
| H₂0 | 74.8 | 74.8 | 74.8 |
| CH₃COOH | 0.7 | 0.7 | 0.7 |
| C₂H₅OH | 1 | 1 | 1 |
| C₂H₄ | 18.8 | 18.8 | 18.8 |
| O₂ | 4.7 | 4.7 | 4.7 |
| CO₂ | 0 | 0 | 0 |

TABLE 15
Example 5. Dry Gas Composition of Feed and Product

| | | Dry Gas Composition (vol. %) | | | | | | |
|---|---|---|---|---|---|---|---|---|
| Exp | | C₂H₂ | C₂H₆ | C₂H₄ | CO | CO₂ | CH₄ | O₂ |
| 1, 2, 3 | Feed | 0.00 | 0.00 | 80.00 | 0.00 | 0.00 | 0.00 | 20.00 |
| 1 | Product | 0.00 | 0.04 | 86.20 | 0.04 | 0.12 | 0.00 | 13.59 |
| 2 | Product | 0.00 | 0.04 | 86.75 | 0.04 | 0.22 | 0.00 | 12.94 |
| 3 | Product | 0.00 | 0.05 | 87.24 | 0.03 | 0.31 | 0.00 | 12.35 |

Note:
each of the product gas composition reported in this table is an average of two sets of experimental data.

TABLE 16
Example 5. Liquid Composition of Feed and Product

| | | Liquid Composition (wt. %) | | | | | |
|---|---|---|---|---|---|---|---|
| | | | | | Acetic | | Other |
| Exp | | H₂O | Methanol | Ethanol | Acid | Acetone | Oxygenates |
| 1, 2, 3 | Feed | 93.86 | 0.15 | 3.25 | 2.74 | 0.00 | 0.00 |
| 1 | Product | 95.37 | 0.09 | 3 | 1.34 | 0 | 0 |
| 2 | Product | * | * | * | * | * | * |
| 3 | Product | * | * | * | * | * | * |

*Not measured.

### Examples Summary of Data

Table 16 below gives the reactor temperature and the feed composition for Examples 1-5. In Table 16, the liquid feed components of water (H₂O), acetic acid (CH₃COOH), and ethanol (C₂H₅OH) were considered as vapor at 22.4 liters per mole. Table 17 give the dry gas composition of the feed and the product for Examples 1-5. Table 18 gives the liquid composition of the feed and product for Examples 1-5. In Table 18, liquid methanol (CH₃OH) is noted as a minor constituent of the feed and product.

TABLE 16
Feed Composition and Reactor Temperature

| | Reactor T | Feed Composition (vol. %) | | | | | | |
|---|---|---|---|---|---|---|---|---|
| | (° C.) | H₂O | CH₃COOH | C₂H₅OH | C₂H₄ | C₂H₆ | O₂ | CO₂ |
| Example 1 | 250 | 70 | 2 | 0 | 23 | 0 | 5 | 0 |
| Example 2 | 350 | 70 | 2 | 0 | 23 | 0 | 5 | 0 |
| Example 3 | 250 | 74 | 2 | 0 | 24 | 0 | 0 | 0 |
| Example 4 | 350 | 7 | 2 | 0 | 0 | 16 | 0 | 70 |
| Example 5 | 325 | 74.8 | 0.7 | 1 | 18.8 | 0 | 4.7 | 0 |
| Example 5 | 334 | 74.8 | 0.7 | 1 | 18.8 | 0 | 4.7 | 0 |
| Example 5 | 340 | 74.8 | 0.7 | 1 | 18.8 | 0 | 4.7 | 0 |

TABLE 17
Dry Gas Composition of Feed and Product

| | Dry Gas Composition (vol. %) | | | | | |
|---|---|---|---|---|---|---|
| | C₂H₆ | C₂H₄ | CO | CO₂ | CH₄ | O₂ |
| Example 1 Feed | 0.00 | 82.14 | 0.00 | 0.00 | 0.00 | 17.86 |
| Example 1 Product | 0.04 | 82.86 | 0.00 | 0.00 | 0.00 | 17.06 |
| Example 2 Feed | 0.00 | 82.14 | 0.00 | 0.00 | 0.00 | 17.86 |
| Example 2 Product | 0.05 | 80.51 | 0.47 | 0.41 | 0.00 | 18.57 |
| Example 3 Feed | 0.00 | 100.00 | 0.00 | 0.00 | 0.00 | 0.00 |
| Example 3 Product | 0.08 | 99.59 | 0.00 | 0.05 | 0.00 | 0.00 |
| Example 4 Feed | 17.58 | 0.00 | 0.00 | 76.92 | 0.00 | 5.49 |
| Example 4 Product | 17.35 | 0.00 | 0.00 | 78.40 | 0.00 | 4.56 |
| Example 5 Feed | 0.00 | 80.00 | 0.00 | 0.00 | 0.00 | 20.00 |
| Example 5 Product | 0.04 | 86.20 | 0.04 | 0.12 | 0.00 | 13.59 |
| Reactor T = 325° C. | | | | | | |
| Example 5 Product | 0.04 | 86.75 | 0.04 | 0.22 | 0.00 | 12.94 |
| Reactor T = 334° C. | | | | | | |
| Example 5 Product | 0.05 | 87.24 | 0.03 | 0.31 | 0.00 | 12.35 |
| Reactor T = 340° C. | | | | | | |

TABLE 18
Liquid Composition of Feed and Product

| | Liquid Composition (wt. %) | | | |
|---|---|---|---|---|
| | H₂O | CH₃OH | C₂H₅OH | CH₃COOH |
| Example 1 Feed | 90.52 | 0 | 0 | 9.48 |
| Example 1 Product | 91.96 | 0 | 0 | 8.02 |
| Example 2 Feed | 89.94 | 0 | 0 | 10.06 |
| Example 2 Product | 88.76 | 0 | 0 | 11.24 |
| Example 3 Feed | 87.59 | 0 | 0 | 12.41 |
| Example 3 Product | 91.11 | 0 | 0 | 8.69 |
| Example 4 Feed | 46.32 | 0 | 0 | 53.68 |
| Example 4 Product | 56.25 | 0 | 0 | 43.75 |
| Example 5 Feed | 93.86 | 0.15 | 3.25 | 2.74 |
| Example 5 Product | 95.37 | 0.09 | 3 | 1.34 |
| Reactor T = 325° C. | | | | |

A number of implementations have been described. Nevertheless, it will be understood that various modifications may be made without departing from the spirit and scope of the disclosure.

### INDUSTRIAL APPLICABILITY

The present disclosure relates to a process for oxidative dehydrogenation to produce ethylene, including an effluent cooling step with short residence times to limit formation of unwanted byproducts downstream of the reactor.

1. A method of operating an oxidative dehydrogenation (ODH) reactor system, the method comprising:
feeding ethane, oxygen, and diluent to an ODH reactor having ODH catalyst;
dehydrogenating ethane to ethylene via the ODH catalyst in presence of the oxygen in the ODH reactor, thereby forming acetic acid in the ODH reactor; and
discharging effluent from the ODH reactor through a quench heat exchanger, thereby cooling the effluent via the quench heat exchanger to below a temperature threshold, the effluent comprising ethylene, acetic acid, water, carbon dioxide, carbon monoxide, and unreacted ethane, wherein residence time of the effluent from the ODH reactor to an outlet of the quench heat exchanger that discharges the effluent is less than a specified upper limit.

2. The method of claim 1, wherein the specified upper limit is specified to decrease occurrence of an unwanted reaction in the effluent.

3. The method of claim 1, comprising specifying the temperature threshold less than 275° C. and above a dew point of the effluent, wherein the specified upper limit is less than 60 seconds.

4. The method of claim 1, comprising specifying the temperature threshold less than 250° C. and above a dew point of the effluent, wherein the specified upper limit is less than 20 seconds.

5. The method of claim 1, comprising conveying, via a conduit, the effluent from an outlet of the ODH reactor to the quench heat exchanger.

6. The method of claim 5, wherein the conduit comprises an internal that decreases residence time of the effluent in the conduit.

7. The method of claim 6, wherein the internal reduces volume of the conduit available for flow of the effluent, thereby decreasing the residence time of the effluent in the conduit, and wherein the internal comprises a static internal.

8. The method of claim 6, wherein the internal comprises a static mixer.

9. The method of claim 1, wherein the quench heat exchanger is attached directly to the ODH reactor.

10. The method of claim 9, wherein attachment of the quench heat exchanger directly to the ODH reactor comprises a flange-to-flange connection comprising a flange of an outlet nozzle of the ODH reactor bolted to a flange of an inlet nozzle of the quench heat exchanger, and wherein the outlet nozzle or the inlet nozzle, or both, comprise an internal, thereby decreasing residence time of the effluent through the outlet nozzle or the inlet nozzle, or both.

11. The method of claim 1, wherein at least a portion of the quench heat exchanger is disposed in the ODH reactor, wherein discharge of the ODH reactor comprises the discharge of the quench heat exchanger, wherein the residence time is zero, and wherein cooling the effluent comprises cooling the effluent via the quench heat exchanger prior to discharging the effluent from the ODH reactor.

12. The method of claim 1, wherein the diluent comprises water, wherein the temperature threshold is in a range of 200° C. to 300° C., and wherein the specified upper limit is less than 60 seconds.

13. A method of operating an oxidative dehydrogenation (ODH) reactor system, the method comprising:
providing feed comprising ethane and oxygen to an ODH reactor;
dehydrogenating ethane to ethylene via ODH catalyst in the ODH reactor; and
discharging effluent from the ODH reactor through a quench heat exchanger, thereby cooling the effluent via the quench heat exchanger to below a specified temperature threshold, the effluent comprising ethylene, acetic acid, water, carbon dioxide, carbon monoxide, and unreacted ethane, wherein residence time of the effluent from an outlet of the ODH reactor that discharges the effluent to an outlet of the quench heat exchanger that discharges the effluent as cooled is less than an upper limit as specified to decrease occurrence of an unwanted reaction in the effluent.

14. The method of claim 13, wherein the upper limit is less than 40 seconds, and wherein the specified temperature threshold is less than 300° C. and above a dew point of the effluent.

15. The method of claim 13, comprising flowing a cooling medium through the quench heat exchanger to cool the effluent via the quench heat exchanger, wherein the quench heat exchanger comprises a shell-and-tube heat exchanger.

16. The method of claim 15, wherein the cooling medium comprises water, and wherein the specified temperature threshold is less than 250° C.

17. The method of claim 15, wherein the cooling medium comprises demineralized water, boiler feedwater, or steam condensate.

18. The method of claim 17, comprising generating steam from the cooling medium via the shell-and-tube heat exchanger with heat from the effluent.

19. The method of claim 13, wherein the quench heat exchanger comprises a heat-pipe heat exchanger, and wherein the temperature threshold is in a range of 200° C. to 300° C.

20. The method of claim 13, comprising flowing the effluent from the quench heat exchanger through a feed heat exchanger comprising a cross exchanger to heat the feed with the effluent, thereby further cooling the effluent, wherein the upper limit is less than 60 seconds.

21. An oxidative dehydrogenation (ODH) reactor system, comprising:
an ODH reactor comprising ODH catalyst to dehydrogenate ethane in presence of oxygen to ethylene and generate acetic acid; and
a quench heat exchanger to cool effluent of the ODH reactor to below a threshold temperature, the effluent comprising ethylene, acetic acid, water, carbon dioxide, carbon monoxide, and unreacted ethane, wherein the ODH reactor system is configured to provide residence time of the effluent from an effluent outlet of the ODH reactor to an effluent outlet of the quench heat exchanger less than an upper limit as specified to decrease occurrence of an unwanted reaction in the effluent.

22. The ODH reactor system of claim 21, wherein the upper limit is less than 60 seconds, and wherein the temperature threshold is less than 300° C.

23. The ODH reactor system of claim 21, wherein the quench heat exchanger comprises a shell-and-tube heat exchanger configured to receive a cooling medium to cool the effluent.

24. The ODH reactor system of claim 23, wherein the shell-and-tube heat exchanger is configured to receive boiler feedwater as the cooling medium to facilitate generation of steam from the boiler feedwater with heat from the effluent.

25. The ODH reactor system of claim 21, wherein the quench heat exchanger comprises a heat-pipe heat exchanger.

26. The ODH reactor system of claim 21, comprising a conduit to convey the effluent from the outlet of the ODH reactor to the quench heat exchanger.

27. The ODH reactor system of claim 26, comprising a static internal disposed in the conduit to reduce flow volume of the conduit to decrease residence time of the effluent in the conduit.

28. The ODH reactor system of claim 21, wherein the quench heat exchanger is directly attached to the ODH reactor.

29. The ODH reactor system of claim 21, wherein the quench heat exchanger is directly attached to the ODH reactor via a flange-to-flange connection in which a flange of an outlet nozzle comprising the outlet of the ODH reactor is bolted to a flange of an inlet nozzle of the quench heat exchanger.

30. The ODH reactor system of claim 29, comprising a static internal in the outlet nozzle or the inlet nozzle, or both, to decrease residence time of the effluent through the outlet nozzle or the inlet nozzle, or both.

31. The ODH reactor system of claim 21, wherein at least a portion of the quench heat exchanger is disposed in the ODH reactor to give the residence time as zero, and wherein to cool the effluent comprises to cool the effluent prior to discharge of the effluent from the ODH reactor.
